(19) United States
(12) Patent Application Publication
(10) Pub. No.: US 2013/0058999 A1
(43) Pub. Date: Mar. 7, 2013

(54) PHARMACEUTICAL COMPOSITIONS FOR ORAL ADMINISTRATION OF INSULIN PEPTIDES

(71) Applicant: Florian Anders Foeger, Frederiksberg C (DK)

(72) Inventor: Florian Anders Foeger, Frederiksberg C (DK)

(73) Assignee: Novo Nordisk A/S, Bagsvaerd (DK)

(21) Appl. No.: 13/521,377

(22) PCT Filed: Jan. 12, 2011
(86) PCT No.: PCT/EP11/50338

§ 371 Date: Nov. 20, 2012

Related U.S. Application Data
(60) Provisional application No. 61/294,621, filed on Jan. 13, 2010.

(30) Foreign Application Priority Data
Jan. 12, 2010 (EP) 10150496.7

(51) Int. Cl. A61K 38/28; A61K 9/48; A61P 3/10; A61K 9/00

(52) U.S. Cl. 424/455; 514/5.9; 424/400

(57) ABSTRACT

The invention is related to pharmaceutical compositions suitable for oral administration of insulin peptides, methods of making such and treatment with such.

### FIELD OF THE INVENTION

The invention is related to pharmaceutical compositions comprising at least one insulin peptide, at least one semi-polar protic organic solvent and at least two non-ionic surfactants, methods of making such and methods of treatment.

### BACKGROUND OF THE INVENTION

Diabetes mellitus is a metabolic disorder in which the ability to utilize glucose is partly or completely lost which may be treated with e.g. insulin.

The general approach for insulin delivery is parenteral administration which is invasive and inconvenient. Therefore non-invasive routes like oral delivery of protein based pharmaceuticals are increasingly investigated. However several barriers exist such as enzymatic degradation in the gastrointestinal (GI) tract, drug efflux pumps, insufficient and variable absorption from the intestinal mucosa, as well as first pass metabolism in the liver. Human insulin is degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

A useful vehicle for oral administration of a drug to a mammal, e.g., a human, is in the form of a microemulsion or nanoemulsion preconcentrate, also called SMEDDS or SNEDDS (self micro or nano emulsifying drug delivery systems), or an emulsion preconcentrate, also called SEDDS (self emulsifying drug delivery systems). SEDDS, SMEDDS or SNEDDS formulations are isotropic mixtures of an oil, a surfactant, a cosurfactant or solubilizer, and any other agents or excipients as needed. When the components of the system come into contact with an aqueous medium, e.g., water, a microemulsion, nanoemulsion or emulsion spontaneously forms, such as an oil-in-water emulsion or microemulsion, with little or no agitation. Microemulsions are thermodynamically stable systems comprising two immiscible liquids, in which one liquid is finely dispersed into the other because of the presence of a surfactant(s). The microemulsion formed, appears to be e.g., clear or translucent, slightly opaque, opalescent, non-opaque or substantially non-opaque because of the low particle size of the dispersed phase.

WO2009115469A1 is related to protease stabilized, acylated insulin analogues and compositions comprising such, WO2003047494A2, U.S. Pat. No. 5,444,041 and WO02094221A1 are related to emulsion/microemulsion compositions, WO9637215A1 is related to insulin water in oil emulsions, US20060210622A1 is related to surface modified particulate compositions of biologically active substances, WO03030865A1, U.S. Pat. No. 5,206,219A and US2004097410A1 are related to insulin compositions including e.g. surfactants and/or lipid components, US20060182771A1 is related to self emulsifying compositions for treatment of ocular diseases and WO2008145730A1, WO2008145728A1 and Ma Er-Li et al., Acta Pharmacologica Sinica, October 2006, Vol. 27, Nr. 10, page 1382-1388, are related to microemulsion or emulsion preconcentrates.

SMEDDS compositions are known to improve the solubility and oral bioavailability of hydrophobic polypeptides such as cyclosporine. However, the solubility of hydrophilic water soluble polypeptides such as human insulin in SMEDDS and SNEDDS is insufficient and bioavailability may not always be optimal. Improved SMEDDS and/or SNEDDS compositions are thus needed for oral delivery of insulins.

### SUMMARY OF THE INVENTION

The present invention is related to liquid non-aqueous pharmaceutical compositions comprising at least one insulin peptide, at least one semi-polar protic organic solvent and at least two non-ionic surfactants with HLB above 10.

In one aspect of the invention, a pharmaceutical composition is described wherein the composition does not contain oil or any other lipid component or surfactant with an HLB below 7.

In one aspect a pharmaceutical composition is described according to the invention, wherein the composition forms a micro- or nanoemulsion after dilution in an aqueous medium.

In another aspect of the invention a pharmaceutical composition is described which comprises two or three non-ionic surfactants with HLB above 10, wherein the remaining ingredients are other excipients than surfactants.

Also methods of producing a pharmaceutical composition according to the invention are described and methods for treatment of hyperglycemia comprising oral administration of an effective amount of a pharmaceutical composition according to the invention.

### DESCRIPTION OF THE DRAWINGS

FIG. 1. Pharmakokinetic profiles of the insulin derivative B29K(N(∈)Octadecanedioyl-γGlu-OEG-OEG) A14E B25H desB30 human insulin (60 nmol/kg) formulated in SMEDDS comprising propylene glycol, Tween 20, Labrasol ALF and diglycerol caprylate after injection into mid-jejunum of anaesthetized overnight fasted Sprague-Dawley rats.

FIG. 2. Pharmakokinetic profiles of the insulin derivative B29K(N(∈)Octadecanedioyl-γGlu-OEG-OEG) A14E B25H desB30 human insulin (60 nmol/kg) formulated in SMEDDS comprising propylene glycol, Tween 20, Labrasol ALF and diglycerol caprylate after injection into mid-jejunum of anaesthetized overnight fasted Sprague-Dawley rats.

FIG. 3. Pharmakokinetic profiles of the insulin derivative B29K(N(∈)Octadecanedioyk-γGlu-OEG-OEG) A14E B25H desB30 human insulin (60 nmol/kg) formulated in SMEDDS or SEDDS comprising propylene glycol, diglycerol caprylate, Tween 20, Plurol Oleique, Labrasol ALF, super refined polysorbate 20 and Rylo MG08 Pharma, after injection into mid-jejunum of fasted male SPRD rats (mean±SEM, n=4-6). SMEDDS comprising 2 or 3 surfactants with HLB above 10 (-□- and -▾-) showed higher plasma insulin levels than formulations comprising at least one lipophilic component (-▪-, -*-) (such as Rylo MG08 or Plurol Oleique) with HLB below 7 or a formulation comprising just one surfactant (-Δ-).

FIG. 4. Pharmakokinetic profiles of the insulin derivative B29K(N(∈)Octadecanedioyk-γGlu-OEG-OEG) A14E B25H desB30 human insulin (60 nmol/kg) formulated in SMEDDS comprising propylene glycol, Tween 20, Labrasol ALF and diglycerol caprylate after injection into mid-jejunum of fasted male SPRD rats (mean±SEM, n=6-7). SMEEDS formulations comprising 2 or 3 surfactants with an HLB above 10 showed significantly higher insulin derivative plasma levels than a formulation comprising just one surfactant and the lipophilic component Rylo MG08 (-x-).

FIG. 5. Pharmakokinetic profiles of insulin derivative B29K(N(∈)Octadecanedioyl-γGlu-OEG-OEG) A14E B25H desB30 human insulin (60 nmol/kg) formulated in SEDDS or SMEDDS after injection into mid-jejunum of fasted male SPRD rats (mean±SEM, n=6-7). SMEDDS formulation comprising 3 surfactants with HLB above 10 (-□-, -▪-) showed higher plasma insulin levels than SMEDDS comprising 2 surfactants or a SEDDS formulation comprising just one surfactant (-x-).

FIG. 6. Pharmacokinetic profiles after per oral dosing of an enteric coated soft capsule comprising insulin derivative B29K(N(∈)Octadecanedioyl-γGlu-OEG-OEG) A14E B25H desB30 human insulin (30 nmol/kg) formulated in SMEDDS (15% PG, 32.5% Labrasol ALF, 32.5% Cremophor RH40, 20% RyloMG08), to male beagle dogs (n=8). Soft capsules were enteric coated with Eudragit L30 D-55.

FIG. 7. Pharmacokinetic profiles after endoscope dosing of uncoated soft capsules comprising insulin derivative B29K(N(eps)Octadecanedioyl-gGlu-OEG-OEG) A14E B25H desB30 human insulin (30 nmol/kg) formulated in SMEDDS (15% propylene glycol, 30% super refined polysorbate 20 and 55% Diglycerol caprylate), to male beagle dogs (n=8). Soft capsules were dosed with an endoscope to the duodenum of beagle dogs.

FIG. 8. Pharmacokinetic profiles after per-oral dosing of coated soft capsules comprising insulin derivative B29K(N(eps)Octadecanedioyl-gGlu-OEG-OEG) A14E B25H desB30 human insulin (45-50 nmol/kg) formulated in SMEDDS (15% propylene glycol, 30% super refined polysorbate 20 and 55% Diglycerol caprylate), to male beagle dogs (n=8). Soft capsules were enteric coated with Eudragit L30 D-55.

FIG. 9. Pharmacokinetic profiles after per-oral dosing of coated soft capsules comprising insulin derivative A14E, B16H, B25H, B29K(N(eps)-Hexadecandioyl-gGlu), desB30 human insulin (30 nmol/kg) formulated in SMEDDS (15% propylene glycol, 30% super refined polysorbate 20 and 55% Diglycerol caprylate), to male beagle dogs (n=8). Soft capsules were enteric coated with a mixture of Eudragit L30 D-55 & Eudragit NE30D.

FIG. 10. Pharmakokinetic profiles of different acylated insulin derivatives (30 nmol/kg) formulated in SMEDDS (15% Propylene glycol, 30% polysorbate 20, 55% diglycerol caprylate) after injection into mid-jejunum of fasted male SPRD rats (mean±SEM, n=6).

FIG. 11. Pharmakokinetic profiles of the insulin derivative B29K(N(eps)OctadecanedioylgGlu-OEG-OEG) A14E B25H desB30 human insulin (60 nmol/kg) formulated in different SMEDDS compositions after injection into mid-jejunum of fasted male SPRD rats (mean±SEM, n=6).

FIG. 12. Pharmakokinetic profiles of the insulin derivative B29K(N(eps)OctadecanedioylgGlu-OEG-OEG) A14E B25H desB30 human insulin (3.25 mg insulin per gram SMEDDS) formulated in a water free SMEDDS compositions and in a SMEDDS composition comprising 5% water, after injection of 0.1 ml into mid-jejunum of fasted male SPRD rats (mean±SEM, n=6).

FIG. 13. Pharmakokinetic profiles of the insulin derivative B29K(N(eps)OctadecanedioylgGlu-OEG-OEG) A14E B25H desB30 human insulin (30 nmol/kg) formulated SMEDDS compositions comprising propylene glycol, Tween 20 and diglycerol caprylate, after injection into mid-jejunum of fasted male SPRD rats (mean±SEM, n=6).

FIG. 14. Pharmakokinetic profiles of different insulin derivatives a), b), c), d), e), f), g), (30 nmol/kg) formulated in a SMEDDS composition according to the invention, comprising 15% propylene glycol, 30% Tween 20 and 55% diglycerol caprylate, after injection into mid-jejunum of fasted male SPRD rats (mean±SEM, n=6).

### DESCRIPTION OF THE INVENTION

The present invention is related to liquid non-aqueous pharmaceutical compositions comprising at least one insulin peptide, at least one semi-polar protic organic solvent and at least two non-ionic surfactants with HLB above 10. In one aspect of the invention, the cornpositions form a microemulsion or nanoemulsion after dilution in an aqueous medium.

It is an important aspect, that the liquid non-aqueous pharmaceutical compositions of the invention comprise at least two non-ionic surfactants with HLB above 10. It has thus surprisingly been found by the inventors that said novel compositions have high oral bioavailability as e.g. compared to known compositions comprising just one surfactant. In one aspect a liquid non-aqueous pharmaceutical composition of the invention comprises at least three non-ionic surfactants with HLB above 10. In one aspect a liquid non-aqueous pharmaceutical composition of the invention comprises two or three non-ionic surfactants with HLB above 10, wherein the remaining ingredients are other excipients than surfactants.

In one aspect a liquid non-aqueous pharmaceutical composition of the invention contains less than 10% oil or any other lipid component or surfactant with an HLB below 7. In one aspect a liquid non-aqueous pharmaceutical composition of the invention contains less than 5% oil or any other lipid component or surfactant with an HLB below 7. In one aspect a liquid non-aqueous pharmaceutical composition of the invention contains less than 1% oil or any other lipid component or surfactant with an HLB below 7.

In one aspect a liquid non-aqueous pharmaceutical composition of the invention comprises two non-ionic surfactants with HLB above 10, wherein the remaining ingredients are other excipients than surfactants. In one aspect a liquid non-aqueous pharmaceutical composition of the invention comprises three non-ionic surfactants with HLB above 10, wherein the remaining ingredients are other excipients than surfactants.

The hydrophilic-lipophilic balance (HLB) of each of the non-ionic surfactants of the liquid non-aqueous pharmaceutical composition of the invention is above 10 whereby high insulin peptide (such as insulin derivative) drug loading capacity and high oral bioavailability are achieved. In one aspect the non-ionic surfactants according to the invention are non-ionic surfactants with HLB above 11. In one aspect the non-ionic surfactants according to the invention are non-ionic surfactants with HLB above 12.

With the term “oral bioavailability” is herein meant the fraction of the administered dose of drug that reaches the systemic circulation after having been administered orally. By definition, when a medication is administered intravenously, its bioavailability is 100%. However, when a medication is administered via other routes, such as orally, its bioavailability decreases due to incomplete absorption and first-pass metabolism. With the term “high oral bioavailability” is thus meant that a high amount of active drug (i.e. the insulin) reaches the systemic circulation after having been administered orally.

As used herein, the term “liquid” means a component or composition that is in a liquid state at room temperature (“RT”), and having a melting point of, for example, below 20° C. As used herein room temperature (RT) means approximately 20-25° C.

In one aspect of the invention the liquid non-aqueous pharmaceutical composition does not contain oil or any other lipid component or surfactant with an HLB below 7. This has the advantage that high amounts of insulin derivatives can be dissolved in these SMEDDS or SNEDDS. In a further aspect the composition does not contain oil or any other lipid component or surfactant with an HLB below 8. In a yet further aspect the composition does not contain oil or any other lipid component or surfactant with an HLB below 9. In a yet further aspect the composition does not contain oil or any other lipid component or surfactant with an HLB below 10.

The liquid non-aqueous pharmaceutical compositions according to the invention comprise at least one semi-polar protic organic solvent. In one aspect the liquid non-aqueous pharmaceutical composition according to the invention comprises only one semi-polar protic organic solvent. In one aspect the semi-polar protic organic solvent according to the invention is a polyol such as e.g. a diol or a triol. In one aspect the semi-polar protic organic solvent is selected from the group consisting of glycerol (propanetriol), ethanediol (ethylene glycol), 1,3-propanediol, methanol, 1,4-butanediol, 1,3-butanediol, propylene glycol (1,2-propanediol), ethanol and isopropanol, or mixtures thereof. In one aspect the semi-polar protic organic solvent according to the invention is selected from the group consisting of propylene glycol, glycerol and mixtures thereof. In one aspect the semi-polar protic organic solvent according to the invention is propylene glycol.

The combination of e.g. propylene glycol and at least two non-ionic surfactants with HLB above 10 in a pharmaceutical composition according to the invention has surprisingly led to a high oral bioavailability of insulin derivatives.

The components of the drug delivery system may be present in any relative amounts. In one aspect the drug delivery system comprises up to 15% polar organic component by weight of the composition of the carrier, i.e. up to 15% of the weight of the carrier consists of the polar organic component before addition of the insulin. In one aspect the drug delivery system comprises from about 1% to about 15% by weight polar organic solvent of the total composition of the carrier. In yet a further aspect, the drug delivery system cornprises from about 5% to about 15% by weight polar organic solvent of the total composition of the carrier. In one aspect, the drug delivery system comprises from about 10% to about 15% by weight polar organic solvent of the total composition of the carrier. In a further aspect, the drug delivery system comprises about 15% by weight polar organic solvent of the total composition of the carrier.

The liquid non-aqueous pharmaceutical compositions according to the invention may have a surprisingly high insulin peptide (such as insulin derivative) drug loading capability, i.e. the compositions may comprise a high amount of insulin. In one aspect of the invention the therapeutically active insulin peptide may be present in an amount up to about 20% such as up to about 10% by weight of the total pharmaceutical composition, or from about 0.1% such as from about 1%. In one aspect of the invention, the therapeutically active insulin peptide may be present in an amount from about 0.1% to about 20%, in a further aspect from about 0.1% to 10%, 0.1% to 20%, 1% to 20% or from about 1% to 10% by weight of the total composition. It is intended, however, that the choice of a particular level of insulin peptide will be made in accordance with factors well-known in the pharmaceutical arts, including the solubility of the insulin peptide in the polar organic solvent or optional hydrophilic component or surfactant used, or a mixture thereof, mode of administration and the size and condition of the patient.

The term “non-aqueous” as used herein refers to a composition to which no water is added during preparation of the pharmaceutical composition. It is known to the person skilled in the art that a composition which has been prepared without addition of water may take up small amounts of water from the surroundings during handling of the pharmaceutical composition such as e.g. a soft-capsule or a hard-capsule used to encapsulate the composition. Also, the insulin peptide and/or one or more of the excipients in the pharmaceutical composition may have small amounts of water bound to it before preparing a pharmaceutical composition according to the invention. A non-aqueous pharmaceutical composition according to the invention may thus contain small amounts of water. In one aspect a non-aqueous pharmaceutical composition according to the invention comprises less than 10% w/w water. In another aspect, the composition according to the invention comprises less than 5% w/w water. In another aspect, the composition according to the invention comprises less than 4% w/w water, in another aspect less than 3% w/w water, in another aspect less than 2% w/w water and in yet another aspect less than 1% w/w water.

When used herein the term “semi-polar protic organic solvent” shall mean a solvent which refers to a hydrophilic, water miscible carbon-containing solvent that contains one or more alcohol or amine functional groups or mixtures thereof. The polarity is reflected in the dielectric constant or the dipole moment of a solvent. The polarity of a solvent determines what type of compounds it is able to dissolve and with what other solvents or liquid compounds it is miscible. Typically, polar solvents dissolve polar compounds best and non-polar solvents dissolve non-polar compounds best: “like dissolves like”. Strongly polar compounds like inorganic salts (e.g. sodium chloride) dissolve only in very polar solvents.

Semi-polar solvents are here defined as solvents with a dielectricity constant in the range of 20-50, whereas polar and non-polar solvents are defined by a dielectricity constant above 50 and below 20, respectively. Examples of semi-polar protic are listed in Table 1 together with water as a reference.

TABLE 1
Dielectricity constants (static permittivity) of selected semi-polar
organic protic solvents and water as a reference (Handbook of
Chemistry and Physics, CMC Press, dielectricity constants are
measured in static electric fields or at relatively low frequencies,
where no relaxation occurs).

| Solvent (Temperature, Kelvin) | Dielectricity constant, ε* |
|---|---|
| Water (293.2) | 80.1 |
| Propanetriol [Glycerol] (293.2) | 46.53 |
| Ethanediol [Ethylene Glycol] (293.2) | 41.4 |
| 1,3-propanediol (293.2) | 35.1 |
| Methanol (293.2) | 33.0 |
| 1,4-butanediol (293.2) | 31.9 |
| 1,3-butanediol (293.2) | 28.8 |
| 1,2-propanediol [propylene glycol] | 27.5 |
| (303.2) | |
| Ethanol (293.2) | 25.3 |
| Isopropanol (293.2) | 20.18 |

In the present context, 1,2-propanediol and propylene glycol is used interchangeably. In the present context, propanetriol and glycerol is used interchangeably. In the present context, ethanediol and ethylene glycol is used interchangeably.

The term “polyol” as used herein refers to chemical compounds containing multiple hydroxyl groups. The term “diol” as used herein refers to chemical compounds containing two hydroxyl groups. The term “Viol” as used herein refers to chemical compounds containing three hydroxyl groups.

The surfactants of the pharmaceutical composition of the invention are nonionic. Surfactants can be complex mixtures containing side products or un-reacted starting products involved in the preparation thereof, e.g., surfactants made by polyoxyethylation may contain another side product, e.g., PEG. The surfactants according to the invention have a hydrophilic-lipophilic balance (HLB) value which is at least 10. For example, the surfactants may have a mean HLB value of 10-30, e.g., 10-20 or 11-17. The surfactants can be liquid, semisolid or solid in nature.

The Hydrophilic-lipophilic balance (HLB) of a surfactant is a measure of the degree to which it is hydrophilic or lipophilic, determined by calculating values for the different regions of the molecule, as described by Griffin (Griffin WC: “Classification of Surface-Active Agents by ‘HLB,’” Journal of the Society of Cosmetic Chemists 1 (1949): 311) or by Davies (Davies JT: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

In one aspect of the invention the nonionic surfactant according to the invention comprise a “medium chain fatty acid group”. A medium chain fatty acid group is herein understood as a fatty acid group having a chain which has from 6 to 12 carbon atoms. In one aspect a medium chain fatty acid group has from 8 to 12 carbon atoms. In one aspect a medium chain fatty acid group is selected from the group consisting of C8 fatty acids (caprylates), C10 fatty acids (caprates) and C12 fatty acids (laurates).

The term “non-ionic surfactant” as used herein refers to any substance, in particular a detergent, that can adsorb at surfaces and interfaces, like liquid to air, liquid to liquid, liquid to container or liquid to any solid and which has no charged groups in its hydrophilic group(s) (sometimes referred to as “heads”). The non-ionic surfactant may be selected from a detergent such as ethoxylated castor oil, polyglycolyzed glycerides, acetylated monoglycerides and sorbitan fatty acid esters, polysorbate such as polysorbate-20, polysorbate-40, polysorbate-60, polysorbate-80, super refined polysorbate 20, super refined polysorbate 40, super refined polysorbate 60 and super refined polysorbate 80 (where the term “super refined” is used by the supplier Croda for their high purity Tween products), poloxamers such as poloxamer 188 and poloxamer 407, polyoxyethylene sorbitan fatty acid esters, polyoxyethylene derivatives such as alkylated and alkoxylated derivatives (Tweens, e.g. Tween-20 or Tween-80), block copolymers such as polyethyleneoxide/polypropyleneoxide block copolymers (e.g. Pluronics/Tetronics, Triton X-100 and/or Synperonic PE/L 44 PEL) and ethoxylated sorbitan alkanoates surfactants (e.g. Tween-20, Tween-40, Tween-80, Brij-35), diglycerol laurate, diglycerol caprate, diglycerol caprylate, diglycerol monocaprylate, polyglycerol laurate, polyglycerol caprate and polyglycerol caprylate.

Examples of other non-ionic surfactants include, but are not limited to:

1. Reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. Various such surfactants are commercially available, e.g., the CREMOPHOR series from BASF Corp. (Mt. Olive, N.J.), such as CREMOPHOR RH 40 which is PEG40 hydrogenated castor oil which has a saponification value of about 50- to 60, an acid value less than about one, a water content, i.e., Fischer, less than about 2%, an n_{D}⁶⁰ of about 1.453-1.457, and an HLB of about 14-16;
2. Polyoxyethylene fatty acid esters that include polyoxyethylene stearic acid esters, such as the MYRJ series from Uniqema e.g., MYRJ 53 having a m.p. of about 47° C. Particular compounds in the MYRJ series are, e.g., MYRJ 53 having an m.p. of about 47° C. and PEG-40-stearate available as MYRJ 52;
3. Sorbitan derivatives that include the TWEEN series from Uniqema, e.g., TWEEN 60;
4. Polyoxyethylene-polyoxypropylene co-polymers and block co-polymers or poloxamers, e.g., Pluronic F127 or Pluronic F68 from BASF or Synperonic PE/L from Croda;
5. Polyoxyethylene alkyl ethers, e.g., such as polyoxyethylene glycol ethers of C₁₂-C₁₈ alcohols, e.g., polyoxyl 10- or 20-cetyl ether or polyoxyl 23-lauryl ether, or 20-oleyl ether, or polyoxyl 10-, 20- or 100-stearyl ether, as known and commercially available as the BRIJ series from Uniqema. Particularly useful products from the BRIJ series are BRIJ 58; BRIJ 76; BRIJ 78; BRIJ 35, i.e. polyoxyl 23 lauryl ether, and BRIJ 98, i.e., polyoxyl 20 oleyl ether. These products have a m.p. between about 32° C. to about 43° C.;
6. Water-soluble tocopheryl PEG succinic acid esters available from Eastman Chemical Co. with a m.p. of about 36° C., e.g, TPGS, e.g., vitamin E TPGS.
7. PEG sterol ethers having, e.g., from 5-35 [CH₂—CH, —O] units, e.g., 20-30 units, e-g., SOLULAN C24 (Choleth-24 and Cetheth-24) from Chemron (Paso Robles, Calif.); similar products which may also be used are those which are known and commercially available as NIKKOL BPS-30 (polyethoxylated 30 phytosterol) and NIKKOL BPSH-25 (polyethoxylated 25 phytostanol) from Nikko Chemicals;
8. Polyglycerol fatty acid esters, e.g., having a range of glycerol units from 4-10, or 4, 6 or 10 glycerol units. For example, particularly suitable are deca-/hexa-/tetraglyceryl monostearate, e.g., DECAGLYN, HEXAGLYN and TETRAGLYN from Nikko Chemicals;
9. Alkylene polyol ether or ester, e.g., lauroyl macrogol-32 glycerides and/or stearoyl macrogol-32 glycerides which are GELUCIRE 44/14 and GELUCIRE 50/13 respectively;
10. Polyoxyethylene mono esters of a saturated C₁₀ to C₂₂, such as C₁₈ substituted e.g. hydroxy fatty acid; e.g. 12 hydroxy stearic acid PEG ester, e.g. of PEG about e.g. 600-900 e.g. 660 Daltons MW, e.g. SOLUTOL HS 15 from BASF (Ludwigshafen, 20 Germany). According to a BASF technical leaflet MEF 151E (1986), SOLUTOL HS 15 comprises about 70% polyethoxylated 12-hydroxystearate by weight and about 30% by weight unesterified polyethylene glycol component. It has a hydrogenation value of 90 to 110, a saponification value of 53 to 63, an acid number of maximum 1, and a maximum water content of 0.5% by weight;
11. Polyoxyethylene-polyoxypropylene-alkyl ethers, e.g. polyoxyethylene-polyoxypropyleneethers of C₁₂ to C₁₈ alcohols, e.g. polyoxyethylen-20-polyoxypropylene-4-cetylether which is commercially available as NIKKOL PBC 34 from Nikko Chemicals;
12. Polyethoxylated distearates, e.g. commercially available under the tradenames ATLAS G 1821 from Uniqema and NIKKOCDS-6000P from Nikko Chemicals.

When used herein the term “Hydrophilic-lipophilic balance” or “HLB” of a surfactant or lipophilic component is a measure of the degree to which it is hydrophilic or lipophilic, determined by calculating values for the different regions of the molecule, as described by Griffin (Griffin WC: “Classification of Surface-Active Agents by ‘HLB,’” Journal of the Society of Cosmetic Chemists 1 (1949): 311) or by Davies (Davies JT: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

“Non-ionic surfactants with HLB above 10” are a selection of non-ionic surfactants which have the common feature of having HLB above 10.

For exemplification, a non-limiting list of surfactants with HLB above 10 is provided below together with their HLB value:

Polyethylene glycol sorbitane monolaurate (e.g. Tween 20, Polysorbate 20, super refined polysorbate 20) with an HLB of 16.7;
Polyoxyethylene (20) sorbitan monooleate (e.g. Tween 80, Polysorbate 80, super refined polysorbate 80) with an HLB of 15;
Polyoxyethylene (20) sorbitan monopalmitate (e.g. Tween 40, Polysorbate 40, super refined polysorbate 40) with an HLB of 15.6;
Diglycerol caprylate (diglycerol monocaprylate, polyglycerol caprylate) with an HLB of 11. Polyglycerol caprate (e.g. Rylo PG10 Pharma) with HLB of 10;
Caprylocaproyl macrogolglycerides (e.g. Labrasol, Labrasol ALF) with an HLB of 14;
Block polymers (e.g. SYNPERONIC PE/L 44, Poloxamer 124);
Polyoxyethylenestearate (e.g. Myrj 45, Macrogolstearate) with HLB of 11.1;
Polyoxyethylenestearate (e.g. Myrj 49, Macrogolstearate) with HLB of 15;
Polyoxyethylenestearate (e.g. Myrj 51, Macrogolstearate) with HLB of 16;
Polyoxyethylenestearate (e.g. Myrj 52, Macrogolstearate) with HLB of 16.9;
Polyoxyethylenestearate (e.g. Myrj 53, Macrogolstearate) with HLB of 17.9;
Polyoxyethylenestearate (e.g. Myrj 59, Macrogolstearate) with HLB of 18.8; and
Polyoxyethyleneglyceroltriricinoleat (e.g. Cremophor EL) with HLB of 13.3.

Examples of liquid non-ionic surfactants with HLB above 10 that may be used in a liquid non-aqueous pharmaceutical composition according to the invention include, but are not limited to, sorbitan derivatives such as TWEEN 20, TWEEN 40 and TWEEN 80, SYNPERONIC L44, and polyoxyl 10-oleyl ether, all available from Uniqema or Croda, and polyoxyethylene containing surfactants e.g. PEG-8 caprylic/capric glycerides (e.g. Labrasol or Labrasol ALF available from Gattefosse).

In one aspect of the invention, one or more of the non-ionic surfactants with HLB above 10 is selected from the group consisting of polyoxyethylene-polyoxypropylene co-polymers, block co-polymers and poloxamers, such as e.g., Pluronic F127, Pluronic F68 from BASF and/or Synperonic from Croda.

In one aspect of the invention, one or more of the non-ionic surfactants with HLB above 10 is a polyoxyethylene containing surfactant such as e.g. PEG-8 caprylic/capric glycerides (e.g. Labrasol or Labrasol ALF available from Gattefosse).

In one aspect of the invention, one or more of the non-ionic surfactants with HLB above 10 is polyethylene glycol sorbitan monolaurate (e.g. Tween 20 available from Merck, Uniqema or Croda). In a further aspect of the invention, one or more of the non-ionic surfactants with HLB above 10 is selected from the group consisting of super refined polysorbates, such as super refined polysorbate 20, 40, 60, and 80 (e.g. commercially available from Croda).

In one aspect of the invention, one or more of the non-ionic surfactants with HLB above 10 is Cremophor RH40 from BASF.

In one aspect of the invention, one or more of the non-ionic surfactants with HLB above 10 is diglycerol monocaprylate or diglycerol caprate (e.g. available from Danisco).

In one aspect of the invention, two of the non-ionic surfactants with HLB above 10 are diglycerol monocaprylate and polysorbate 20 (e.g. Tween 20).

In the aspect of the invention where the liquid non-aqueous pharmaceutical composition of the invention comprises two non-ionic surfactants with HLB above 10, the two non-ionic surfactants are diglycerol monocaprylate and polysorbate 20 (e.g. Tween 20).

The composition of the invention may comprise from about 30% to about 90% non-ionic surfactants by weight of the composition of the carrier, i.e. from about 30% to about 90% of the weight of the carrier before addition of the insulin consists of the non-ionic surfactants such as e.g. from about 40% to about 85% by weight, e.g., about 50% to about 85% by weight, e.g. from about 60% to about 85% by weight, or e.g. from about 70% to about 85%.

In certain aspects of the present invention, the pharmaceutical composition may comprise additional excipients commonly found in pharmaceutical compositions, examples of such excipients include, but are not limited to, antioxidants, antimicrobial agents, enzyme inhibitors, stabilizers, preservatives, flavors, sweeteners and other components as described in Handbook of Pharmaceutical Excipients, Rowe et al., Eds., 4th Edition, Pharmaceutical Press (2003), which is hereby incorporated by reference.

These additional excipients may be in an amount from about 0.05-5% by weight of the total pharmaceutical composition. Antioxidants, anti-microbial agents, enzyme inhibitors, stabilizers or preservatives typically provide up to about 0.05-1% by weight of the total pharmaceutical composition. Sweetening or flavoring agents typically provide up to about 2.5% or 5% by weight of the total pharmaceutical composition.

In one aspect of the invention, the composition comprises a buffer. The term “buffer” as used herein refers to a chemical compound in a pharmaceutical composition that reduces the tendency of pH of the composition to change over time as would otherwise occur due to chemical reactions. Buffers include chemicals such as sodium phosphate, TRIS, glycine and sodium citrate.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “stabilizer” as used herein refers to chemicals added to peptide containing pharmaceutical compositions in order to stabilize the peptide, i.e. to increase the shelf life and/or in-use time of such compositions.

The quality of non-ionic surfactants suitable for the invention as obtained from the manufacturer may influence the stability of the pharmaceutical composition comprising said non-ionic surfactants. For example certain excipients with higher purity have been identified which stabilize the liquid non-aqueous pharmaceutical composition. It is thus an aspect of the invention that a liquid non-aqueous pharmaceutical composition is obtained wherein the non-ionic surfactant is a high purity non-ionic surfactant. In one aspect a high purity non-ionic surfactant is a non-ionic surfactant which is supplied by the supplier as pharma grade. In one aspect a high purity non-ionic surfactant is a non-ionic surfactant which is supplied by the supplier as super refined. In one aspect a high purity non-ionic surfactant is a non-ionic surfactant which has an aldehyde and/or ketone content below 20 ppm. In another aspect a high purity non-ionic surfactant is a non-ionic surfactant which has an aldehyde and/or ketone content below 10 ppm. In one aspect the non-ionic surfactant is selected from the group consisting of: Diglycerol monocaprylate or diglycerol caprate from Danisco). In another aspect the non-ionic surfactant are polysorbates such as e.g. Tween 20, Tween 80, super refined polysorbate 20, super refined polysorbate 80 from Croda.

The term “oil or any other lipid component or surfactant with an HLB below 7” is used herein for a selection of oils or any other lipid components surfactants which have the common feature of having HLB below 7.

Examples of oils or any other lipid components or surfactants with HLB below 7 include, but are not limited to:

Polyglycerol oleate (e.g. Plurol Oleique CC497) with HLB of 6;
Polyglyceryl-3 Oleate (e.g. Caprol 3GO; Isolan GO33, Triglycerol mono-oleate) with HLB of 5 to 6.5;
Propylene glycol monocaprylate (Capryol 90, Capryol PGMC, Capmul PG) with HLB of 6;
Propylene glycol monolaurate (Lauroglycol 90, Lauroglycol FCC) with HLB of 5;
Propylene glycol dicaprylocaprate (e.g. Labrafac PG) with HLB of 2;
Medium chain triglycerides (i.e. triglycerides with chains having from 8 to 12 carbon atoms, such as 8, 10 or 12 carbon atoms) with HLB of 1 (e.g. Labrafac Lipophile WL1349; Captex 355);
Glyceryl monolinoleate (e.g. Maisine 35-1) with HLB of 4;
Glyceryl monooleate (e.g. Peceol) with HLB of 3;
Lauroyl macrogolglycerides (e.g. Labrafil M2130CS) with HLB of 4;
Linoleoyl macrogolglycerides (e.g. Labrafil M2125CS) with HLB of 4;
Oleoyl macrogolglycerides (e.g. Labrafil M1944CS) with HLB of 4;
Medium chain mono-, di- and/or triglycerides (i.e. mono-, di- and/or triglycerides with chains having from 8 to 12 carbon atoms, such as 8, 10 or 12 carbon atoms) with HLB 5-6 (e.g. Capmul MCM);
Mixed diesters of caprylic/capric acids in propylene glycol (e.g. Captex 200);
Propylene glycol dicaprate ester (e.g. Captex 100); and
Glycerol monocaprate/caprylate (e.g. Rylo MG10 Pharma, Rylo MG8 Pharma) with HLB 6-7.

In certain aspects of the present invention, the pharmaceutical composition may be coated with a coating agent commonly found for pharmaceutical compositions such as oral pharmaceutical compositions. Known coatings e.g. include sugar-coatings, film-catings, polymer and polysaccharide based coatings e.g. with plasticizers and pigments included, coatings comprising opaque materials such as titanium dioxide, coatings having pearlescent effects, controlled-release coatings and enteric coatings. The pharmaceutical composition may be filled into a capsule, e.g. enteric coated capsule, soft capsule, hard capsule or enteric soft capsule.

In one embodiment, the coating comprises at least one release modifying polymer which can be used to control the site where the drug (insulin derivative) is released. The modified release polymer can be a polymethacrylate polymer such as those sold under the Eudragit® trade name (Evonik Rohm GmbH, Darmstadt, Germany), for example Eudragit L30 D55, Eudragit L100-55, Eudragit L100, Eudragit S100, Eudragit S12.5, Eudragit FS30D, Eudragit NE30D and mixtures thereof as e.g. described in Eudragit Application Guidelines, Evonik Industries, 11th edition, September 2009.

As used herein, the term “microemulsion preconcentrate” means a composition, which spontaneously forms a microemulsion or a nanoemulsion, e.g., an oil-in-water microemulsion or nanoemulsion, swollen micelle, micellar solution, in an aqueous medium, e.g. in water or in the gastrointestinal fluids after oral application. The composition self-emulsifies upon dilution in an aqueous medium for example in a dilution of 1:5, 1:10, 1:50, 1:100 or higher.

“SEDDS” (self emulsifying drug delivery systems) are herein defined as mixtures of a hydrophilic component, a surfactant, optionally a cosurfactant and a drug that forms spontaneously a fine oil in water emulsion when exposed to aqueous media under conditions of gentle agitation or digestive motility that would be encountered in the GI tract.

“SMEDDS” (self micro-emulsifying drug delivery systems) are herein defined as isotropic mixtures of a hydrophilic component, a surfactant, optionally a cosurfactant and a drug that rapidly form an oil in water microemulsion or nanoemulsion when exposed to aqueous media under conditions of gentle agitation or digestive motility that would be encountered in the GI tract.

“SNEDDS” (self nano-emulsifying drug delivery systems) are herein defined as isotropic mixtures of a hydrophilic component, at least one surfactant with HLB above 10, optionally a cosurfactant and a drug that rapidly form a nanoemulsion (droplet size below 20 nm in diameter as e.g. measured by PCS) when exposed to aqueous media under conditions of gentle agitation or digestive motility that would be encountered in the GI tract.

As used herein, the term “emulsion” refers to a slightly opaque, opalescent or opague colloidal coarse dispersion that is formed spontaneously or substantially spontaneously when its components are brought into contact with an aqueous medium.

As used herein, the term “microemulsion” refers to a clear or translucent, slightly opaque, opalescent, non-opaque or substantially non-opaque colloidal dispersion that is formed spontaneously or substantially spontaneously when its components are brought into contact with an aqueous medium.

A microemulsion is thermodynamically stable and contains homogenously dispersed particles or domains, for example of a solid or liquid state (e.g., liquid lipid particles or droplets), of a mean diameter of less than 150 nm as measured by standard light scattering techniques, e.g., using a MALVERN ZETASIZER Nano ZS. In one aspect when the pharmaceutical composition according to the invention is brought into contact with an aqueous medium a microemulsion is formed which contains homogenously dispersed particles or domains of a mean diameter of less than 100 nm, such as less than 50 nm, less than 40 nm and less than 30 nm.

The term “domain size” as used herein refers to repetitive scattering units and may be measured by e.g., small angle X-ray. In one aspect of the invention, the domain size is smaller than 150 nm, in another aspect, smaller than 100 nm and in another aspect, smaller than 50 nm, in another aspect, smaller than 20 nm, in another aspect, smaller than 15 nm, in yet another aspect, smaller than 10 nm.

As used herein, the term “nanoemulsion” refers to a clear or translucent, slightly opaque, opalescent, non-opaque or substantially non-opaque colloidal dispersion with particle or droplet size below 20 nm in diameter (as e.g. measured by PCS) that is formed spontaneously or substantially spontaneously when its components are brought into contact with an aqueous medium. In one aspect when the pharmaceutical composition according to the invention is brought into contact with an aqueous medium a microemulsion is formed which contains homogenously dispersed particles or domains of a mean diameter of less than 20 nm, such as less than 15 nm, less than 10 nm and greater than about 2-4 nm.

In one aspect the pharmaceutical composition of the invention forms, when brought into contact with an aqueous medium, a microemulsion with domains below 100 nm in diameter when measured by Photon Correlation Spectroscopy (PCS). PCS is also known as Dynamic Light Scattering (DLS). The time decay of the near-order of the particles caused by the Brownian motion is used to evaluate the size of nanoparticles via the Stokes-Einstein relation. At constant temperature, T, the method only requires the knowledge of the viscosity, h, of the suspending fluid for an estimation of the average particle size and its distribution function (and for volume fractions the refractive index, n).

In one aspect when the pharmaceutical composition according to the invention is brought into contact with an aqueous medium a nanoemulsion is formed.

As used herein the term “spontaneously dispersible” when referring to a preconcentrate refers to a composition that is capable of producing colloidal structures such as nanoemulsions, microemulsions, emulsions and other colloidal systems, when diluted with an aqueous medium when the components of the composition of the invention are brought into contact with an aqueous medium, e.g. by simple shaking by hand for a short period of time, for example for ten seconds. In one aspect a spontaneously dispersible concentrate according to the invention is a SEDDS, SMEDDS or SNEDDS.

The pharmaceutical composition according to the invention is in liquid form.

As used herein, the term “liquid” means a component or composition that is in a liquid state at room temperature (“RT”), and having a melting point of, for example, below 20° C. As used herein room temperature (RT) means approximately 20-25° C.

In one aspect the liquid non-aqueous pharmaceutical composition according to the invention is in liquid form at refrigerated temperature such as about 4° C.

The term “about” as used herein means in reasonable vicinity of the stated numerical value, such as plus or minus 10%.

The liquid non-aqueous pharmaceutical compositions of the invention are both physically and chemically stable, i.e. the shelf life of said compositions is sufficient for being suitable as a drug composition and the pharmaceutical compositions are thus shelf-stable.

The term “shelf-stable pharmaceutical composition” as used herein means a pharmaceutical composition which is stable for at least the period which is required by regulatory agencies in connection with therapeutic proteins. Preferably, a shelf-stable pharmaceutical composition is stable for at least one year at 5° C. Shelf-stability includes chemical stability as well as physical stability. Chemical instability involves degradation of covalent bonds, such as hydrolysis, racemization, oxidation or crosslinking. Chemical stability of the formulations is evaluated by means of reverse phase (RP-HPLC) and size exclusion chromatography SE-HPLC). In one aspect of the invention, the formation of peptide related impurities during shelf-life is less than 20% of the total peptide content. In a further aspect of the invention, the formation of peptide related during impurities during shelf-life is less than 10%. In a further aspect of the invention, the formation of peptide related during impurities during shelf-life is less than 5%. The RP-HPLC analysis is typically conducted in water-acetonitrile or waterethanol mixtures. In one aspect, the solvent in the RP-HPLC step will comprise a salt such as Na₂SO₄, (NH₄)₂SO₄, NaCl, KCl, and buffer systems such as phosphate, and citrate and maleic acid. The required concentration of salt in the solvent may be from about 0.1 M to about 1 M, preferable between 0.2 M to 0.5 M, most preferable between 0.3 to 0.4 M. Increase of the concentration of salt requires an increase in the concentration of organic solvent in order to achieve elution from the column within a suitable time. Physical instability involves conformational changes relative to the native structure, which includes loss of higher order structure, aggregation, fibrillation, precipitation or adsorption to surfaces. Peptides such as insulin peptides, GLP-1 compounds and amylin compounds are known to be prone to instability due to fibrillation. Physical stability of the formulations may be evaluated by conventional means of e.g. visual inspection and nephelometry after storage of the formulation at different temperatures for various time periods. Conformational stability may be evaluated by circular dichroism and NMR as described by e.g. Hudson and Andersen, Peptide Science, vol 76 (4), pp. 298-308 (2004).

The biological activity of an insulin peptide may be measured in an assay as known by a person skilled in the art as e.g. described in WO 2005/012347.

In one aspect of the invention the pharmaceutical composition according to the invention is stable for more than 6 weeks of usage and for more than 3 years of storage.

In another aspect of the invention the pharmaceutical composition according to the invention is stable for more than 4 weeks of usage and for more than 3 years of storage.

In a further aspect of the invention the pharmaceutical composition according to the invention is stable for more than 4 weeks of usage and for more than two years of storage.

In an even further aspect of the invention the pharmaceutical composition according to the invention is stable for more than 2 weeks of usage and for more than two years of storage.

In an even further aspect of the invention the pharmaceutical composition according to the invention is stable for more than 1 weeks of usage and for more than one year of storage.

In one aspect, the pharmaceutical composition according to the invention is used for the preparation of a medicament for the treatment or prevention of hyperglycemia, type 2 diabetes, impaired glucose tolerance, and type 1 diabetes.

With “insulin peptide”, “an insulin peptide” or “the insulin peptide” as used herein is meant human insulin with disulfide bridges between CysA7 and CysB7 and between CysA20 and CysB19 and an internal disulfide bridge between CysA6 and CysA11 or an insulin analogue or derivative thereof.

Human insulin consists of two polypeptide chains, the A and B chains which contain 21 and 30 amino acid residues, respectively. The A and B chains are interconnected by two disulphide bridges. Insulin from most other species is similar, but may contain amino acid substitutions in some positions.

An insulin analogue as used herein is a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue.

In one aspect an insulin analogue according to the invention comprises less than 8 modifications (substitutions, deletions, additions) relative to human insulin. In one aspect an insulin analogue comprises less than 7 modifications (substitutions, deletions, additions) relative to human insulin. In one aspect an insulin analogue comprises less than 6 modifications (substitutions, deletions, additions) relative to human insulin. In another aspect an insulin analogue comprises less than 5 modifications (substitutions, deletions, additions) relative to human insulin. In another aspect an insulin analogue comprises less than 4 modifications (substitutions, deletions, additions) relative to human insulin. In another aspect an insulin analogue comprises less than 3 modifications (substitutions, deletions, additions) relative to human insulin. In another aspect an insulin analogue comprises less than 2 modifications (substitutions, deletions, additions) relative to human insulin.

An insulin derivative according to the invention is a naturally occurring insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin.

An insulin derivative is thus human insulin or an insulin analogue which comprises at least one covalent modification such as a side-chain attached to one or more amino acids of the insulin peptide.

Herein, the naming of the insulin peptide is done according to the following principles: The names are given as mutations and modifications (acylations) relative to human insulin. With “desB30 human insulin” is thus meant an analogue of human insulin lacking the B30 amino acid residue. Similarly, “desB29desB30 human insulin” means an analogue of human insulin lacking the B29 and B30 amino acid residues. With “B1”, “A1” etc. is meant the amino acid residue at position 1 in the B-chain of insulin (counted from the N-terminal end) and the amino acid residue at position 1 in the A-chain of insulin (counted from the N-terminal end), respectively. The amino acid residue in a specific position may also be denoted as e.g. PheB1 which means that the amino acid residue at position B1 is a phenylalanine residue.

In one aspect an insulin derivative for use in a pharmaceutical composition of the invention has a side chain attached either to the α-amino group of the N-terminal amino acid residue of B chain or to an ∈-amino group of a Lys residue present in the B chain of the insulin peptide via an amide bond

In one aspect the side chain comprises at least one OEG group. In one aspect the side chain comprises a fatty diacid moiety with 4 to 22 carbon atoms. In one aspect the side chain comprises at least one free carboxylic acid group or a group which is negatively charged at neutral pH. In one aspect the side chain comprises at least one linker which links the individual components in the side chain together via amide, ether or amine bonds, said linkers optionally comprising a free carboxylic acid group.

In one aspect the side chain comprises at least one OEG group, a fatty diacid moiety with 4 to 22 carbon atoms, at least one free carboxylic acid group or a group which is negatively charged at neutral pH and optionally at least one linker which links the individual components in the side chain together via amide, ether or amine bonds, said linkers optionally comprising a free carboxylic acid group.

In one aspect of the invention the side chain comprises from 1 to 20 OEG groups; from 1 to 10 OEG groups or from 1 to 5 OEG groups.

In one aspect, an insulin derivative in a non-aqueous pharmaceutical composition according to the invention is an insulin peptide that is acylated in one or more amino acids of the insulin peptide.

In one aspect, an insulin derivative in a non-aqueous pharmaceutical composition according to the invention is an insulin peptide that is acylated via an amide bond to the α-amino group of the N-terminal amino acid residue of B chain and/or the ∈-amino group of one or more Lys residues present in the B chain of the insulin peptide.

For the naming of the acyl moiety, the naming is done according to IUPAC nomenclature and in other cases as peptide nomenclature. For example, naming the acyl moiety:

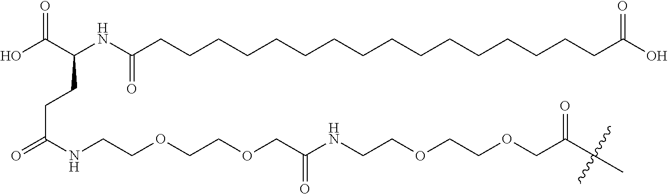 [SMILES: *C(=O)COCCOCCNC(=O)COCCOCCNC(=O)CC[C@H](NC(=O)CCCCCCCCCCCCCCCCC(=O)O)C(=O)O]

can be e.g. “octadecanedioyl-γ-L-Glu-OEG-OEG”, or “17-carboxyheptadecanoyl-γ-L-Glu-OEG-OEG”, wherein OEG is short hand notation for —NH(CH₂)₂—O—(CH₂)₂OCH₂CO—, and γ-L-Glu (or g-L-Glu) is short hand notation for the L-form of the amino acid gamma glutamic acid moiety.

The acyl moiety of the modified peptides or proteins may be in the form of a pure enantiomer wherein the stereo configuration of the chiral amino acid moiety is either D or L (or if using the R/S terminology: either R or S) or it may be in the form of a mixture of enanti-omers (D and L/R and S). In one aspect of the invention the acyl moiety is in the form of a mixture of enantiomers. In one aspect the acyl moiety is in the form of a pure enantiomer. In one aspect the chiral amino acid moiety of the acyl moiety is in the L form. In one aspect the chiral amino acid moiety of the acyl moiety is in the D form.

In one aspect, an insulin derivative in a non-aqueous pharmaceutical composition according to the invention is an insulin peptide that is stabilized towards proteolytic degradation (by specific mutations) and further acylated at the B29-lysine. A non-limiting example of insulin peptides that are stabilized towards proteolytic degradation (by specific mutations) may e.g. be found in WO 2008/034881, which is hereby incorporated by reference.

The acylated insulin peptides of this invention may be mono-substituted having only one acylation group attached to a lysine amino acid residue in the protease stabilized insulin molecule.

In one aspect, the insulin peptide is acylated to either the α-amino group of the N-terminal amino acid residue of the B chain or an ∈-amino group of a Lys residue present in the B chain of the insulin peptide. In one aspect, the insulin peptide is acylated to the ∈-amino group of a Lys residue present in position B29 of the insulin peptide.

A non-limiting list of acylated insulin peptides suitable for the liquid non-aqueous pharmaceutical composition of the invention may e.g. be found in WO 2009/115469 such as in the passage beginning on page 25 thereof and continuing the next 6 pages.

In one aspect, the insulin derivative in a non-aqueous liquid pharmaceutical composition according to the invention is an acylated insulin which is found in WO 2009/115469, such as the acylated insulins listed in claim 8 in WO 2009/115469.

In one aspect of the invention, the acylated insulin peptide is selected from the group consisting of:

B29K(N(∈)hexadecanedioyl-γ-L-Glu) A14E B25H desB30 human insulin;

B29K(N(∈)octadecanedioyl-γ-L-Glu-OEG-OEG) desB30 human insulin;

B29K(N(∈)octadecanedioyl-γ-L-Glu) A14E B25H desB30 human insulin;

B29K(N(∈)eicosanedioyl-γ-L-Glu) A14E B25H desB30 human insulin;

B29K(N(∈)octadecanedioyl-γ-L-Glu-OEG-OEG) A14E B25H desB30 human insulin;

B29K(N(∈)eicosanedioyl-γ-L-Glu-OEG-OEG) A14E B25H desB30 human insulin;

B29K(N(∈)eicosanedioyl-γ-L-Glu-OEG-OEG) A14E B16H B25H desB30 human insulin;

B29K(N(∈)hexadecanedioyl-γ-L-Glu) A14E B16H B25H desB30 human insulin;

B29K(N(∈)Octadecanedioyl-γ-L-Glu) A14E B25H desB27 desB30 human insulin;

B29K(N(∈)Octadecanedioyl-γ-L-Glu-OEG-OEG) A14E B25H desB27 desB30 human insulin;

B29K(N(∈)eicosanedioyl-γ-L-Glu-OEG-OEG) A14E B16H B25H desB30 human insulin; and

B29K(N(∈)octadecanedioyl) A14E B25H desB30 human insulin.

In another aspect of the invention, the insulin derivative is B29K(N(∈)octadecanedioyl-γ-L-Glu-OEG-OEG) A14E B25H desB30 human insulin.

The insulin peptide may be present in an amount up to about 20% such as up to about 10% by weight of the total pharmaceutical composition, or from about 0.1% such as from about 1%. In one aspect of the invention, the insulin peptide is present in an amount from about 0.1% to about 20%, in a further aspect from about 0.1% to 15%, 0.1% to 10%, 1% to 8% or from about 1% to 5% by weight of the total composition. It is intended, however, that the choice of a particular level of insulin peptide will be made in accordance with factors well-known in the pharmaceutical arts, including the solubility of the insulin peptide in the polar organic solvent or optional hydrophilic component or surfactant used, or a mixture thereof, mode of administration and the size and condition of the patient.

Each unit dosage will suitably contain from 1 mg to 200 mg insulin peptide, e.g. about 1 mg, 2 mg, 5 mg, 10 mg, 15 mg, 25 mg, 50 mg, 80 mg, 90 mg, 100 mg, 150 mg, 200 mg insulin peptide, e.g. between 5 mg and 200 mg of insulin peptide. In one aspect of the invention each unit dosage contains between 10 mg and 200 mg of insulin peptide. In a further aspect a unit dosage form contains between 10 mg and 100 mg of insulin peptide. In yet a further aspect of the invention, the unit dosage form contains between 20 mg and 80 mg of insulin peptide. In yet a further aspect of the invention, the unit dosage form contains between 30 mg and 60 mg of insulin peptide. In yet a further aspect of the invention, the unit dosage form contains between 30 mg and 50 mg of insulin peptide. Such unit dosage forms are suitable for administration 1-5 times daily depending upon the particular purpose of therapy.

The production of polypeptides and peptides such as insulin is well known in the art. Polypeptides or peptides may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides or peptides may also be produced by a method which comprises culturing a host cell containing a DNA sequence encoding the (poly)peptide and capable of expressing the (poly)peptide in a suitable nutrient medium under conditions permitting the expression of the peptide. For (poly)peptides comprising non-natural amino acid residues, the recombinant cell should be modified such that the non-natural amino acids are incorporated into the (poly)peptide, for instance by use of tRNA mutants.

In yet a further aspect, the invention provides a process for preparing a pharmaceutical composition such as SEDDS, SMEDDS or SNEDDS (which may be filled into a capsule, e.g. enteric coated capsule, soft capsule or enteric soft capsule) containing an insulin peptide, which process comprises the following steps:

(a) dissolving first the insulin peptide in the polar organic solvent (such as propylene glycol) and

(b) then mixing with the non-ionic surfactants and optionally additional components.

In one aspect of the present invention, a process for preparing the pharmaceutical composition is carried out at low temperature (e.g. room temperature or below room temperature).

When preparing the pharmaceutical composition according to the invention, the insulin peptide may e.g. be dissolved in the polar organic solvent using the following method:

a) providing an aqueous solution of the insulin peptide optionally comprising excipients,

b) adjusting the pH value to a target pH value which is 1 unit, alternatively 2 units and alternatively 2.5 pH units above or below the pI of the insulin peptide,

c) removing water from (dehydrating) the insulin peptide by conventional drying technologies such as freeze- or spray drying, and

d) mixing and dissolving the insulin peptide in said polar non-aqueous solvent e.g. by stirring, tumbling or other mixing methods,

e) optionally filtering or centrifuging the non-aqueous insulin peptide solution to remove non-dissolved inorganic salts,

f) optionally removing residual amounts of waters by e.g. adding solid dessicants or vacuum drying.

In one aspect the insulin peptide is dissolved in the polar organic solvent by the following method:

a) providing an aqueous solution of an insulin peptide, optionally containing stabilizers such as zinc and glycylglycine,

b) adjusting the pH value to 1 unit, alternatively 2 units and alternatively 2.5 pH units above or below the pI of the insulin peptide e.g. by adding a non-volatile base or a acid, such as hydrochloric acid or sodium hydroxide, to the solution

c) removing water from (dehydrating) the insulin peptide by conventional drying technologies such as freeze- and spray drying,

d) mixing and dissolving of the insulin peptide in said polar non-aqueous solvent e.g. by stirring, tumbling or other mixing methods,

e) optionally filtering or centrifuging the non-aqueous insulin peptide solution to remove non-dissolved inorganic salts,

f) optionally removing residual amounts of waters by e.g. adding solid dessicants or vacuum drying.

By “volatile base” is meant a base, which to some extend will evaporate upon heating and/or at reduced pressure, e.g. bases which have a vapour pressure above 65 Pa at room temperature or an aqueous azeotropic mixture including a base having a vapour pressure above 65 Pa at room temperature. Examples of volatile bases are ammonium hydroxides, tetraalkylammonium hydroxides, secondary amines, tertiary amines, aryl amines, alphatic amines or ammonium bicarbonate or a combination. For example the volatile base may be bicarbonate, carbonate, ammonia, hydrazine or an organic base such as a lower aliphatic amines e.g. trimethyl amine, triethylamine, diethanolamines, triethanolamine and their salts. Further the volatile base may be ammonium hydroxide, ethyl amine or methyl amine or a combination hereof.

By “volatile acid” is meant an acid, which to some extend will evaporate upon heating and/or at reduced pressure, e.g. acids which have a vapour pressure above 65 Pa at room temperature or an aqueous azeotropic mixture including an acid having a vapour pressure above 65 Pa at room temperature. Examples of volatile acids are carbonic acid, formic acid, acetic acid, propionic acid and butyric acid.

A “non volatile base” as mentioned herein means a base, which does not evaporate or only partly evaporate upon heating, e.g. bases with a vapour pressure below 65 Pa at room temperature. The non volatile base may be selected from the group consisting of alkaline metal salts, alkaline metal hydroxides, alkaline earth metal salts, alkaline earth metal hydroxides and amino acids or a combination hereof. Examples of non-volatile bases are sodium hydroxide, potassium hydroxide, calcium hydroxide, and calcium oxide.

A “non volatile acid” as mentioned herein means an acid, which does not evaporate or only partly evaporate upon heating, e.g. bases with a vapour pressure below 65 Pa at room temperature. Examples of non-volatile acids are hydrochloric acid, phosphoric acid and sulfuric acid.

In one aspect an insulin peptide according to the invention is soluble in propylene glycol. In another aspect an insulin peptide according to the invention is soluble in a propylene glycol solution comprising at least 20% w/w insulin peptide. In yet another aspect of the invention a insulin peptide according to the invention is soluble in a propylene glycol solution comprising at least 30% w/w insulin peptide.

In one aspect of the present invention, the insulin peptide is pH optimized before dissolution in the polar organic solvent to improve solubility in the polar organic solvent.

When using the term “pH optimized” it is herein meant that the insulin peptide has been dehydrated at a target pH which is at least 1 pH unit from the pI of the insulin peptide in aqueous solution. Thus, in one aspect of the invention, the target pH is more than 1 pH unit above the isoelectric point of the insulin peptide. In another aspect of the invention, the target pH is more than 1 pH unit below the isoelectric point of the insulin peptide. In a further aspect, the target pH is more than 1.5 pH units above or below the pI of the insulin peptide. In a yet further aspect, the target pH is 2.0 pH units or more above or below the pI of the insulin peptide. In a still further aspect, the target pH is 2.5 pH units or more above or below the pI of the insulin peptide. In yet a further aspect, the target pH is above the pI of the insulin peptide.

The term “dehydrated” as used herein in connection with a insulin peptide refers to a insulin peptide which has been dried from an aqueous solution. The term “target pH” as used herein refers to the aqueous pH which will establish when dehydrated insulin peptide is rehydrated in pure water to a concentration of approximately 40 mg/ml or more. The target pH will typically be identical to the pH of the aqueous insulin peptide solution from which the insulin peptide was recovered by drying. However, the pH of the insulin peptide solution will not be identical to the target pH, if the solution contains volatile acids or bases. It has been found that the pH history of the insulin peptide will be determinant for the amount of the insulin peptide, which may be solubilized in the polar organic solvent.

The term “the pI of the insulin peptide” as used herein refers to the isoelectric point of a insulin peptide.

The term “isoelectric point” as used herein means the pH value where the overall net charge of a macromolecule such as a peptide is zero. In peptides there may be several charged groups, and at the isoelectric point the sum of all these charges is zero. At a pH above the isoelectric point the overall net charge of the peptide will be negative, whereas at pH values below the isoelectric point the overall net charge of the peptide will be positive.

The pI of a protein may be determined experimentally by electrophoresis techniques such as electrofocusing:

A pH gradient is established in an anticonvective medium, such as a polyacrylamide gel. When a peptide is introduced in to the system it will migrate under influence of an electric field applied across the gel. Positive charged peptides will migrate to the cathode. Eventually, the migrating peptide reaches a point in the pH gradient where its net electrical charge is zero and is said to be focused. This is the isoelectric pH (pI) of the peptide. The peptide is then fixed on the gel and stained. The pI of the peptide may then be determined by comparison of the position of the peptide on the gel relative to marker molecules with known pI values.

The net charge of a peptide at a given pH value may be estimated theoretically per a person skilled in the art by conventional methods. In essence, the net charge of peptide is the equivalent to the sum of the fractional charges of the charged amino acids in the peptide: aspartate (β-carboxyl group), glutamate (δ-carboxyl group), cysteine (thiol group), tyrosine (phenol group), histidine (imidazole side chains), lysine (∈-ammonium group) and arginine (guanidinium group). Additonally, one should also take into account charge of peptide terminal groups (α-NH2 and α-COOH). The fractional charge of the ionisable groups may be calculated from the intrinsic pKa values.

The drying i.e. dehydration of the insulin peptide may be performed by any conventional drying method such e.g. by spray-, freeze-, vacuum-, open- and contact drying. In one aspect of the invention, the insulin peptide solution is dried to obtain a water content below about 10%. The water content may be below about 8%, below about 6%, below about 5%, below about 4%, below about 3%, below about 2% or below about 1% calculated on/measured by loss on drying test (gravimetric) as stated in the experimental part.

In one aspect of the invention the insulin peptide is spray dried. In a further aspect of the invention, the insulin peptide is freeze-dried.

The Following is a Non-Limiting List of Aspect Further Comprised within the Scope of the Invention:
1. A liquid pharmaceutical composition comprising at least one insulin peptide, at least one semi-polar protic organic solvent and at least two non-ionic surfactants with HLB above 10.
2. A pharmaceutical composition according to aspect 1, wherein the composition does not contain oil or any other lipid component or surfactant with an HLB below 7.
3. A pharmaceutical composition according to aspect 1 or 2, which comprises less than 10% w/w water.
4. A pharmaceutical composition according to anyone of aspects 1-3, which is non-aqueous.
5. A pharmaceutical composition according to anyone of aspects 1 or 3-4, wherein the remaining ingredients are other excipients than surfactants.
6. A pharmaceutical composition according to anyone of aspects 1-5, wherein the composition forms a micro- or nanoemulsion after dilution in an aqueous medium.
7. A pharmaceutical composition according to anyone of the previous aspects wherein the composition forms an emulsion with a droplet size below 100 nm in diameter after 100 fold dilution in an aqueous medium.
8. A pharmaceutical composition according to aspects 6 or 7, wherein the droplet size is analysed by dynamic light scattering.
9. A pharmaceutical composition according to anyone of the previous aspects, wherein the composition does not contain oil or any other lipid component or surfactant with an HLB below 8.
10. A pharmaceutical composition according to anyone of the previous aspects, wherein the composition does not contain oil or any other lipid component or surfactant with an HLB below 9.
11. A pharmaceutical composition according to anyone of the previous aspects, wherein the composition does not contain oil or any other lipid component or surfactant with an HLB below 10.
12. A pharmaceutical composition according to anyone of the previous aspects, wherein said at least two non-ionic surfactants have an HLB above 11.
13. A pharmaceutical composition according to anyone of the previous aspects, wherein said at least two non-ionic surfactants have an HLB above 12.
14. A pharmaceutical composition according to anyone of the previous aspects, comprising at least three non-ionic surfactants with HLB above 10, alternatively with HLB above 11 or alternatively with HLB above 12.
15. A pharmaceutical composition according to anyone of the previous aspects, comprising two or three non-ionic surfactants with HLB above 10, alternatively with HLB above 11 or, alternatively with HLB above 12, wherein the remaining ingredients are other excipients than surfactants.
16. A pharmaceutical composition according to aspect 15, comprising two non-ionic surfactants with HLB above 10, alternatively with HLB above 11 or alternatively with HLB above 12, wherein the remaining ingredients are other excipients than surfactants.
17. A pharmaceutical composition according to aspect 15, comprising three non-ionic surfactants with HLB above 10, alternatively with HLB above 11 or alternatively with HLB above 12, wherein the remaining ingredients are other excipients than surfactants.
18. A pharmaceutical composition according to anyone of the previous aspects, wherein the semi-polar protic organic solvent is a protic solvent with a dielectricity constant in the range of 20-50.
19. A pharmaceutical composition according to anyone of the previous aspects, wherein the semi-polar protic organic solvent is a polyol.
20. A pharmaceutical composition according to anyone of the previous aspects, wherein the semi-polar protic organic solvent is glycerol or propylene glycol.
21. A pharmaceutical composition according to anyone of the previous aspects, wherein the semi-polar protic organic solvent is propylene glycol.
22. A pharmaceutical composition according to anyone of the previous aspects, wherein said non-ionic surfactants are liquid at room temperature
23. A pharmaceutical composition according to anyone of the previous aspects, which is in the form of a solution
24. A pharmaceutical composition according to anyone of the previous aspects, wherein said non-ionic surfactant does not comprise any long chain fatty acid group (e.g. free long chain fatty acids or long chain fatty acid esters) which has from 16 to 20 carbon atoms.
25. A pharmaceutical composition according to anyone of the previous aspects, wherein one or more of said non-ionic surfactants comprise a medium chain fatty acid group
26. A pharmaceutical composition according to anyone of the previous aspects, wherein one or more of said non-ionic surfactants comprise a fatty acid group which has up to 12 carbon atoms.
27. A pharmaceutical composition according to aspect 25, wherein the medium chain fatty acid group has from 6 to 12 carbon atoms
28. A pharmaceutical composition according to aspect 25 or 27, wherein the medium chain fatty acid group has from 8 to 12 carbon atoms
29. A pharmaceutical composition according to aspect 25, 27 or 28, wherein the medium chain fatty acid group is selected from the group consisting of: C8 fatty acids (caprylates), C10 fatty acids (caprates) or C12 fatty acids (laurates)
30. A pharmaceutical composition according to anyone of the previous aspects, wherein one or more of said non-ionic surfactants are selected from the group consisting of Labrasol (also named Caprylocaproyl Macrogolglycerides), Tween 20 (also named Polysorbate 20 or Polyethylene glycol sorbitan monolaurate), Tween 80 (also named polysorbate 80), Diglycerol monocaprylate, Polyglycerol caprylate and Cremophor RH 40.
31. A pharmaceutical composition according to anyone of the previous aspects, wherein said non-ionic surfactants are selected from the group consisting of: Tween 20, Tween 80, Diglycerol monocaprylate and Polyglycerol caprylate.
32. A pharmaceutical composition according to anyone of the previous aspects, wherein one of said non-ionic surfactants is Diglycerol monocaprylate
33. A pharmaceutical composition according to anyone of the previous aspects, wherein the semi-polar protic organic solvent is present in the amount from about 1% to about 15%
34. A pharmaceutical composition according to anyone of the previous aspects, wherein the semi-polar protic organic solvent is present in the amount from about 5% to about 15%
35. A pharmaceutical composition according to anyone of the previous aspects, wherein the semi-polar protic organic solvent is present in the amount from about 10% to about 15%
36. A pharmaceutical composition according to anyone of aspects 1-3 or 5-36, which comprises less than 5% w/w water.
37. A pharmaceutical composition according to aspect 36, which comprises less than 2% w/w water.
38. A pharmaceutical composition according to aspect 37, which comprises less than 1% w/w water.
39. A pharmaceutical composition according to anyone of the previous aspects, wherein the insulin peptide is an insulin derivative.
40. A pharmaceutical composition according to anyone of the previous aspects, wherein the insulin derivative is an acylated insulin peptide.
41. A pharmaceutical composition according to anyone of the previous aspects, wherein the insulin derivative is a protease stabilized insulin which has been derivatized in one or more positions.
42. A pharmaceutical composition according to anyone of the previous aspects, wherein the insulin derivative is a protease stabilized insulin which has been acylated in one or more positions.

43. A pharmaceutical composition according to anyone of the previous aspects, wherein the insulin derivative is mono-substituted having only one acylation group attached to a lysine amino acid residue in the insulin molecule.

44. A pharmaceutical composition according to anyone of the previous aspects, wherein the insulin peptide is acylated to either the α-amino group of the N-terminal amino acid residue of the B chain or an ∈-amino group of a Lys residue present in the B chain of the insulin peptide.
45. A pharmaceutical composition according to anyone of the previous aspects, wherein the insulin peptide is acylated to the ∈-amino group of a Lys residue present in position B29 of the insulin peptide.
46. A pharmaceutical composition according to anyone of aspects 39-45, wherein the insulin derivative is a protease stabilized insulin which has an acyl moiety attached to the protease stabilized insulin, wherein the acyl moiety has the general formula:

Acy-AA1ₙ-AA2ₘ-AA3ₚ- (I),

wherein n is 0 or an integer in the range from 1 to 3;

m is 0 or an integer in the range from 1 to 10;

p is 0 or an integer in the range from 1 to 10;

Acy is a fatty acid or a fatty diacid comprising from about 8 to about 24 carbon atoms;
AA1 is a neutral linear or cyclic amino acid residue;
AA2 is an acidic amino acid residue;
AA3 is a neutral, alkyleneglycol-containing amino acid residue;
and wherein the order by which AA1, AA2 and AA3 appears in the formula can be interchanged independently.
47. A pharmaceutical composition according to aspect 46, wherein n is 0
48. A pharmaceutical composition according to anyone of aspects 46-47, wherein m is an interger in the range from 1 to 10, such as 1 to 5, such as 1.
49. A pharmaceutical composition according to anyone of aspects 46-48, wherein p is an interger in the range from 1 to 10, such as 1 to 5, 1 to 4, 1 to 3, 1 or 2.
50. A pharmaceutical composition according to anyone of aspects 46-49, wherein AA3 is OEG.
51. A pharmaceutical composition according to anyone of aspects 39-50, wherein the insulin derivative selected from the group consisting of:

B29K(N(∈)hexadecanedioyl-γ-L-Glu) A14E B25H desB30 human insulin;

B29K(N(∈)octadecanedioyl-γ-L-Glu-OEG-OEG) desB30 human insulin;

B29K(N(∈)octadecanedioyl-γ-L-Glu) A14E B25H desB30 human insulin;

B29K(N(∈)eicosanedioyl-γ-L-Glu) A14E B25H desB30 human insulin;

B29K(N(∈)octadecanedioyl-γ-L-Glu-OEG-OEG) A14E B25H desB30 human insulin;

B29K(N(∈)eicosanedioyl-γ-L-Glu-OEG-OEG) A14E B25H desB30 human insulin;

B29K(N(∈)eicosanedioyl-γ-L-Glu-OEG-OEG) A14E B16H B25H desB30 human insulin;

B29K(N(∈)hexadecanedioyl-γ-L-Glu) A14E B16H B25H desB30 human insulin;

B29K(N(∈)Octadecanedioyl-γ-L-Glu) A14E B25H desB27 desB30 human insulin;

B29K(N(∈)Octadecanedioyl-γ-L-Glu-OEG-OEG) A14E B25H desB27 desB30 human insulin;

B29K(N(∈)eicosanedioyl-γ-L-Glu-OEG-OEG) A14E B16H B25H desB30 human insulin; and

B29K(N(∈)octadecanedioyl) A14E B25H desB30 human insulin.

52. A pharmaceutical composition according to anyone of the previous aspects, wherein the insulin derivative is soluble in propylene glycol.
53. A pharmaceutical composition according to anyone of the previous aspects, wherein the insulin derivative is soluble in a propylene glycol solution comprising at least 20% w/w insulin derivative.
54. A pharmaceutical composition according to anyone of the previous aspects, wherein the insulin derivative is soluble in a propylene glycol solution comprising at least 30% w/w insulin derivative.
55. A pharmaceutical composition according to anyone of the previous aspects, wherein the insulin peptide is dissolved in the SMEDDS or SNEDDS
56. A pharmaceutical composition according to anyone of the previous aspects, wherein the composition forms a microemulsion with domains below 100 nm in diameter when measured by PCS.
57. A pharmaceutical composition according to anyone of the previous aspects, wherein the composition forms a microemulsion with domains below 50 nm in diameter when measured by PCS.
58. A pharmaceutical composition according to anyone of the previous aspects, wherein the composition forms a microemulsion with domains below 40 nm in diameter when measured by PCS.
59. A pharmaceutical composition according to anyone of the previous aspects, wherein the composition forms a microemulsion with domains below 30 nm in diameter when measured by PCS.
60. A pharmaceutical composition according to anyone of the previous aspects, wherein the composition forms a nanoemulsion with domains below 20 nm in diameter when measured by PCS.
61. A pharmaceutical composition according to anyone of the previous aspects, wherein the composition forms a nanoemulsion with domains below 15 nm in diameter when measured by PCS.
62. A pharmaceutical composition according to anyone of the previous aspects, wherein each other excipient has an HLB above 10, alternatively 11 or alternatively 12.
63. A pharmaceutical composition according to anyone of the previous aspects, further comprising an aldehyde scavenger such as ethylene diamine
64. A pharmaceutical composition according to anyone of the previous aspects, which is encapsulated in a capsule such as a soft capsule or a hard capsule.
65. A pharmaceutical composition according aspect 64, wherein the hard or soft capsule is enteric coated.
66. A method of producing a pharmaceutical composition according to anyone of the previous aspects.
67. A method of producing a pharmaceutical composition according to anyone of the previous aspects comprising the steps of:

(a) dissolving the insulin derivative in the polar organic solvent and

(b) subsequently mixing with the lipophilic component and optionally with the surfactant and/or hydrophilic component.

68. A method of producing a pharmaceutical composition according to anyone of the previous aspects, wherein the method comprises the steps:

a) The insulin is dehydrated at a target pH which is at least one pH unit from the pI of the polypeptide in aqueous solution,

b) the dehydrated insulin is dissolved in the semi polar protic solvent,

c) at least two non ionic surfactants with an HLB above 10 are added together or stepwise under agitation,

d) encapsulation of the liquid formulation into soft capsules or filling into hard capsules,

e) optional enteric coating of the softcapsules or hardcapsules.

69. A pharmaceutical composition according to anyone of aspects 1-65 for use as a medicament.
70. A pharmaceutical composition according to anyone of aspects 1-65 for use as a medicament in the treatment of hyperglycemia.
71. A method for treatment of hyperglycemia comprising oral administration of an effective amount of a pharmaceutical composition as defined in anyone of the aspects 1-65.

### EXAMPLES

### Preparation of Insulin Liquid Non-Aqueous Pharmaceutical Composition:

25 mg of insulin derivative B29K(N(∈)Octadecanedioyl-γGlu-OEG-OEG) A14E B25H desB30 human insulin were dissolved in MilliQ water and the pH was adjusted with NaOH to obtain a pH of 7 to 8. In the next step, the solution was frozen and freeze dried to obtain a neutral insulin powder which was then dissolved in 150 mg of propylene glycol under gentle agitation at RT and under nitrogen. After complete dissolution, 550 mg of diglycerol caprylate were added under gentle agitation at RT under nitrogen. In the final step, 300 mg of polysorbate 20 (Tween 20) were added under agitation at RT under nitrogen. The final liquid composition was clear and homogenously.

Similarly insulin liquid non-aqueous pharmaceutical compositions were prepared with other ingredients.

### Example 1

### Liquid Non-Aqueous Pharmaceutical Composition Comprising Insulin Derivative, Propylene Glycol, Tween 20, Labrasol ALF and Diglycerol Caprylate

Pharmakokinetic profiles were made of the insulin derivative B29K(N(∈)Octadecanedioyl-γGlu-OEG-OEG) A14E B25H desB30 human insulin (60 nmol/kg) formulated in SMEDDS comprising propylene glycol, Tween 20, Labrasol ALF and diglycerol caprylate after injection into mid-jejunum of anaesthetized overnight fasted Sprague-Dawley rats. The insulin derivative was first dissolved in propylene glycol and thereafter the according amounts of the surfactants Tween 20, diglycerol caprylate and Labrasol ALF were added and mixed to obtain a homogenous liquid formulation.

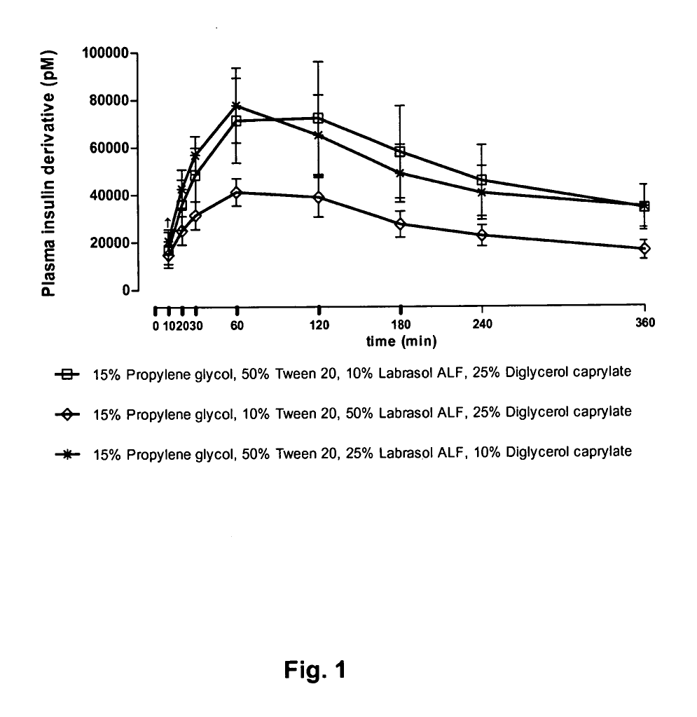

The results are shown in FIG. 1.

### Example 2

### Particle Size Distribution of Emulsions from the Pharmaceutical Compositions of Example 1

The insulin derivative SEDDS, SMEDDS and SNEDDS pharmaceutical compositions described in Example 1 were diluted 50 fold with MilliQ water and the particle size distribution of the resulting emulsions, microemulsions or nanoemulsions were analysed by PCS (DLS) with a Malvem Zetasizer Nano ZS at 37° C. Insulin derivative SMEDDS pharmaceutical compositions resulting in micro- or nanoemulsions showed higher insulin plasma levels than a formulation resulting in a crude emulsion.

Results are shown in Table 2 and FIG. 1.

TABLE 2

| | Z-average size | Intensity PSD | | |
|---|---|---|---|---|
| | (d. nm) | (d. nm) | | PDI |
| 15% propylene glycol, | 8.1 nm | 9.2 | nm (100%) | 0.11 |
| 50% Tween 20, 10% | | | | |
| Labrasol ALF and | | | | |
| 25% diglycerol caprylate | | | | |
| 15% propylene glycol, | 271 nm | >2000 | nm | 1.00 |
| 10% Tween 20, 50% | | | | |
| Labrasol ALF and | | | | |
| 25% diglycerol caprylate | | | | |
| 15% propylene glycol, | 9.2 nm | 9.1 | nm | 0.25 |
| 50% Tween 20, 25% | | | | |
| Labrasol ALF and | | | | |
| 10% diglycerol caprylate | | | | |

PDI: Poly Dispersity Index;
PSD: Particle Size Distribution;
d. nm: diameter in nanometers

### Example 3

### Liquid Non-Aqueous Pharmaceutical Composition Comprising Insulin Derivative, Propylene Glycol, Tween 20, Labrasol ALF and Diglycerol Caprylate

Pharmakokinetic profiles were made of the insulin derivative B29K(N(∈)Octadecanedioyl-γGlu-OEG-OEG) A14E B25H desB30 human insulin (60 nmol/kg) formulated in SMEDDS comprising propylene glycol, Tween 20, Labrasol ALF and diglycerol caprylate after injection into mid-jejunum of anaesthetized overnight fasted Sprague-Dawley rats. The insulin derivative was first dissolved in propylene glycol and thereafter the according amounts of the surfactants Tween 20, diglycerol caprylate and Labrasol ALF were added and mixed to obtain a homogenous liquid formulation.

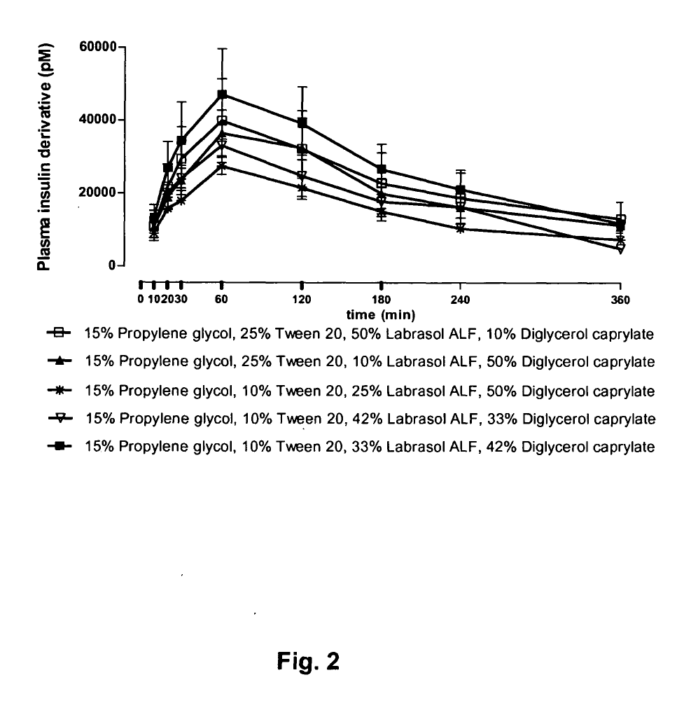

The results are shown in FIG. 2.

### Example 4

### Liquid Non-Aqueous Pharmaceutical Compositions Comprising Insulin Derivative, Propylene Glycol One or More Surfactants

Pharmakokinetic profiles were made of the insulin derivative B29K(N(∈)Octadecanedioyl-γGlu-OEG-OEG) A14E B25H desB30 human insulin (60 nmol/kg) formulated in SMEDDS or SEDDS comprising propylene glycol, diglycerol caprylate, Tween 20, Plurol Oleique, Labrasol ALF, super refined polysorbate 20 and Rylo MG08 Pharma, after injection into mid-jejunum of fasted male SPRD rats (mean±SEM, n=4-6). SMEDDS comprising 2 or 3 surfactants with HLB above 10 showed higher plasma insulin levels than formulations comprising at least one lipophilic component (such as Rylo MG08 or Plurol Oleique) with HLB below 7 or a formulation comprising just one surfactant. The insulin derivative was first dissolved in propylene glycol and thereafter the according amounts of the surfactants or lipophilic components were added and mixed to obtain a homogenous liquid formulation.

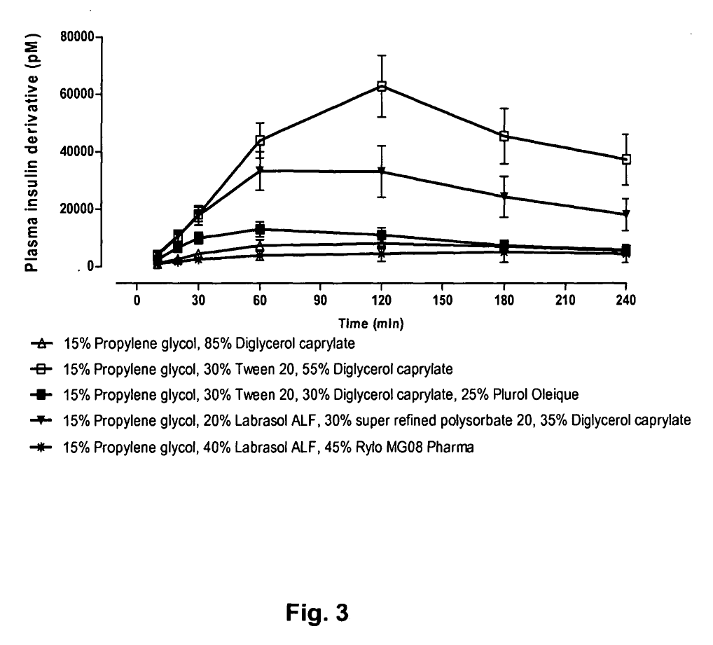

The results are shown in FIG. 3.

### Example 5

### Particle Size Distribution of Emulsions from the Pharmaceutical Compositions of Example 4

Insulin derivative SEDDS, SMEDDS and SNEDDS were diluted 50 fold with MilliQ water and the particle size distribution of the resulting emulsions, microemulsions or nanoemulsions were analysed by PCS (DLS) with a Malvem Zetasizer Nano ZS at 37° C. Insulin derivative SNEDDS resulting in nanoemulsions showed higher insulin plasma levels than SEDDS resuiting in crude emulsions.

Results are shown in Table 3 and FIG. 3.

TABLE 3

| | Z-average size | | Intensity PSD | | |
|---|---|---|---|---|---|
| | (d. nm) | | (d. nm) | | PSD |
| 15% propylene glycol, 30% | 9.9 | nm | 11.0 | nm | 0.10 |
| Tween 20, 55% diglycerol | | | | | |
| caprylate | | | | | |
| 15% propylene glycol, 20% | 10.6 | nm | 11.5 | nm | 0.06 |
| Labrasol ALF, 30% super | | | | | |
| refined polysorbate 20, 35% | | | | | |
| diglycerol caprylate | | | | | |
| 15% propylene glycol, 30% | 85.7 | nm | 142 | nm | 0.42 |
| Tween 20, 30% diglycerol | | | | | |
| caprylate, 25% Plurol | | | | | |
| Oleique | | | | | |
| 15% propylene glycol, 40% | 739 | nm | 467 | nm | 0.79 |
| Labrasol ALF, 45% | | (emulsion) | | | |
| Rylo MG08 Pharma | | | | | |
| 15% propylene glycol, 85% | 3023 | nm | 4854 | nm | 0.68 |
| diglycerol caprylate | | (emulsion) | | | |

PDI: Poly Dispersity Index;
PSD: Particle Size Distribution;
d. nm: diameter in nanometers

### Example 6

### Liquid Non-Aqueous Pharmaceutical Compositions Comprising Insulin Derivative, Propylene Glycol Two or Three Surfactants

Pharmakokinetic profiles were made of the insulin derivative B29K(N(∈)Octadecanedioyl-γGlu-OEG-OEG) A14E B25H desB30 human insulin (60 nmol/kg) formulated in SMEDDS comprising propylene glycol, Tween 20, Labrasol ALF and diglycerol caprylate after injection into mid-jejunum of fasted male SPRD rats (mean±SEM, n=6-7). SMEEDS formulations comprising 2 or 3 surfactants with an HLB above 10 showed significantly higher insulin derivative plasma levels than a formulation comprising just one surfactant and the lipophilic component Rylo MG08. The insulin derivative was first dissolved in propylene glycol and thereafter the according amounts of the surfactants or lipophilic component were added and mixed to obtain a homogenous liquid formulation.

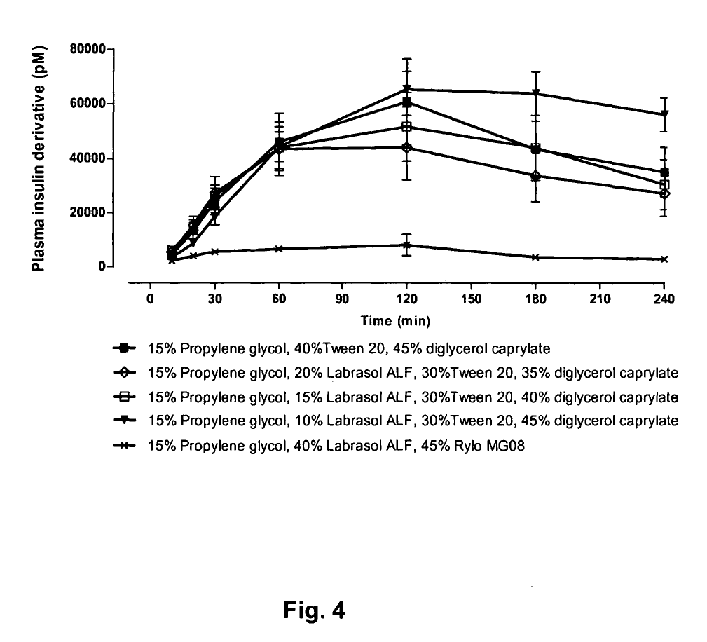

The results are shown in FIG. 4.

### Example 7

### Liquid Non-Aqueous Pharmaceutical Compositions Comprising Insulin Derivative, Propylene Glycol One, Two or Three Surfactants

Pharmakokinetic profiles were made of insulin derivative B29K(N(∈)Octadecanedioyl-γGlu-OEG-OEG) A14E B25H desB30 human insulin (60 nmol/kg) formulated in SEDDS or SMEDDS after injection into mid-jejunum of fasted male SPRD rats (mean±SEM, n=6-7). SMEDDS formulation comprising 3 surfactants with HLB above 10 showed higher plasma insulin levels than SMEDDS comprising 2 surfactants or a SEDDS formulation comprising just one surfactant. The insulin derivative was first dissolved in propylene glycol and thereafter the according amounts of the surfactants or lipophilic component were added and mixed to obtain a homogenous liquid formulation.

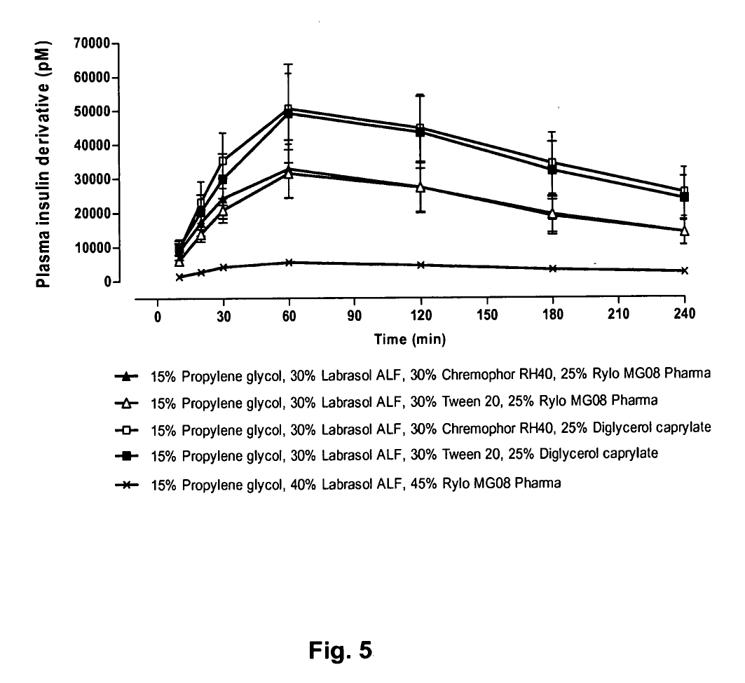

The results are shown in FIG. 5.

### Example 8

### Particle Size Distribution of Emulsions from the Pharmaceutical Compositions of Example 7

Insulin derivative SEDDS, SMEDDS and SNEDDS were diluted 50 fold with MilliQ water and the particle size distribution of the resulting emulsions, microemulsions or nanoemulsions were analysed by PCS (DLS) with a Malvem Zetasizer Nano ZS at 37° C. Insulin derivative SNEDDS resulting in nanoemulsions showed higher insulin plasma levels than SMEDDS resulting in microemulsions or SEDDS resulting in a crude emulsion.

Results are shown in Table 4 and FIG. 5.

TABLE 4

| | Z-average | Intensity | |
|---|---|---|---|
| | size | PSD | |
| | (d. nm) | (d. nm) | PDI |
| 15% propylene glycol, 30% | 11 nm | 12 nm | 0.09 |
| Labrasol ALF, 30% Chremophor | | | |
| RH40, 25% diglycerol caprylate | | | |
| 15% propylene glycol, 30% | 11 nm | 12 nm | 0.17 |
| Labrasol ALF, 30% Tween 20, | | | |
| 25% diglycerol caprylate, | | | |
| 15% propylene glycol, 30% | 49 nm | 60 nm | 0.17 |
| Labrasol ALF, 30% Tween 20, | | | |
| 25% Rylo MG08 Pharma | | | |
| 15% propylene glycol, 30% | 37 nm | 47 nm | 0.19 |
| Labrasol ALF, 30% Chremophor | | | |
| RH40, 25% Rylo MG08 Pharma | | | |
| 15% propylene glycol, 40% | Crude | Crude | Crude |
| Labrasol ALF, 45% Rylo | emulsion | emulsion | emulsion |
| MG08 Pharma | | | |

PDI: Poly Dispersity Index;
PSD: Particle Size Distribution;
d. nm: diameter in nanometers

### Example 9

### Liquid Non-Aqueous Pharmaceutical Compositions Comprising Insulin Derivative, Propylene Glycol, Labrasol ALF, Cremophor RH40 and RyloMG08

Pharmacokinetic profiles were made after per oral dosing of an enteric coated soft capsule comprising insulin derivative B29K(N(∈)OctadecanedioykyGlu-OEG-OEG) A14E B25H desB30 human insulin (30 nmol/kg) formulated in SMEDDS (15% propylene glycol, 32.5% Labrasol ALF, 32.5% Cremophor RH40, 20% RyloMG08), to male beagle dogs (n=8). The insulin derivative was first dissolved in propylene glycol and thereafter the according amounts of the surfactants were added and mixed to obtain a homogenous liquid formulation. The liquid formulation was filled into soft capsules and enteric coated with Eudragit L30D-55.

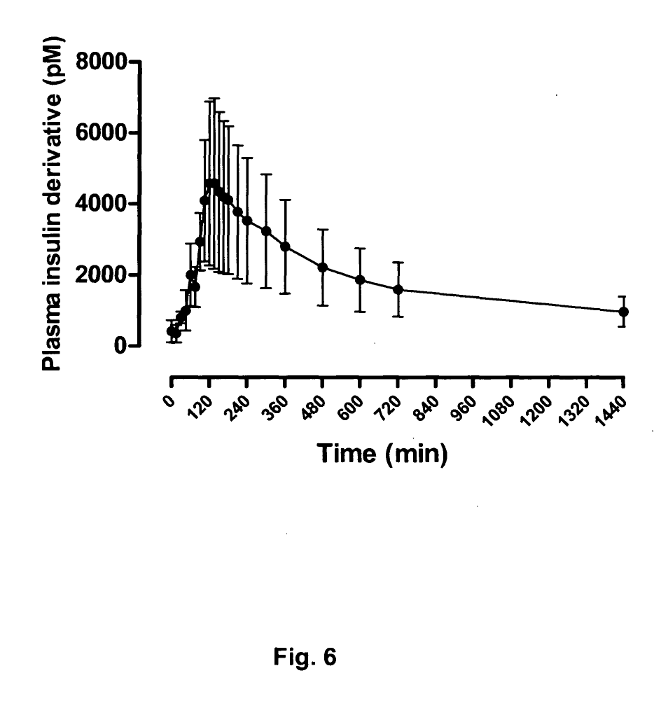

The results are shown in FIG. 6.

### Example 10

### Uncoated Soft Capsule Comprising Insulin Derivative Formulated in SMEDDS

Pharmacokinetic profiles were made after endoscope dosing of uncoated soft capsules comprising insulin derivative B29K(N(eps)Octadecanedioyl-gGlu-OEG-OEG) A14E B25H desB30 human insulin (30 nmol/kg) formulated in SMEDDS (15% propylene glycol, 30% super refined polysorbate 20 and 55% Diglycerol caprylate), to male beagle dogs (n=8). Soft capsules were dosed with an endoscope to the duodenum of male beagle dogs.

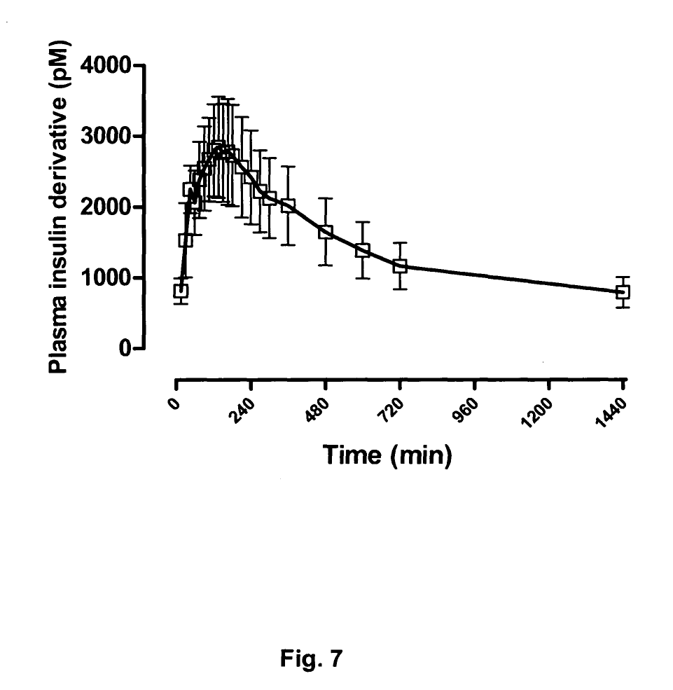

The results are shown in FIG. 7

### Example 11

### Enteric Soft Capsules Comprising Insulin Derivative Formulated in SMEDDS

Pharmacokinetic profiles were made after per-oral dosing of coated soft capsules comprising insulin derivative B29K(N(eps)Octadecanedioyl-gGlu-OEG-OEG) A14E B25H desB30 human insulin (45-50 nmol/kg) formulated in SMEDDS (15% propylene glycol, 30% super refined polysorbate 20 and 55% Diglycerol caprylate), to male beagle dogs (n=8). Soft capsules were enteric coated with Eudragit L30 D-55.

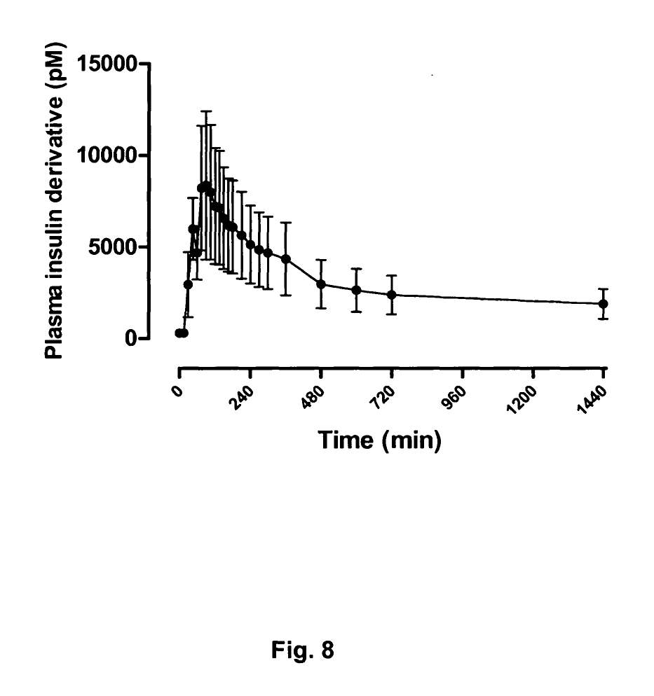

The results are shown in FIG. 8.

### Example 12

### Enteric Soft Capsules Comprising Insulin Derivative Formulated in SMEDDS

Pharmacokinetic profiles were made after per-oral dosing of enteric coated soft capsules comprising insulin derivative A14E, B16H, B25H, B29K(N(eps)-Hexadecandioyl-gGlu), desB30 human insulin (30 nmol/kg) formulated in SMEDDS (15% propylene glycol, 30% super refined polysorbate 20 and 55% Diglycerol caprylate), to male beagle dogs (n=8). Soft capsules were enteric coated with a 1:1 mixture of Eudragit L30 D-55 and Eudragit NE30D.

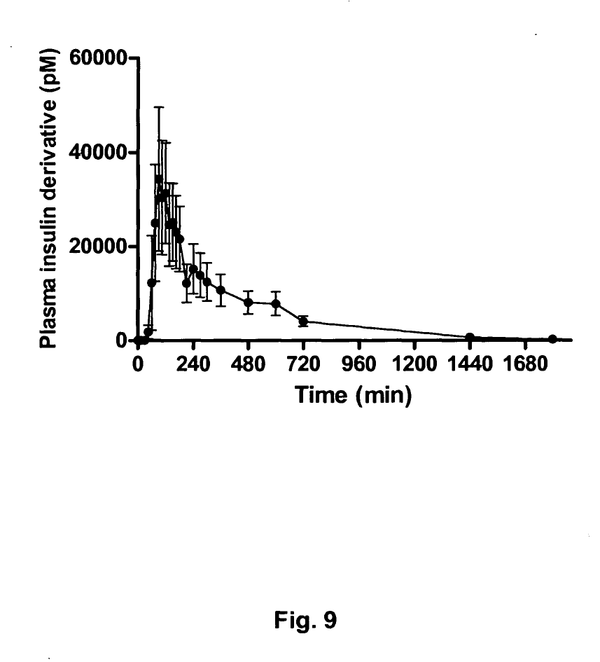

The results are shown in FIG. 9.

### Example 13

### Different Insulin Derivatives Formulated in SMEDDS

Pharmakokinetic profiles of different acylated insulin derivatives (30 nmol/kg) formulated in SMEDDS (15% Propylene glycol, 30% polysorbate 20, 55% diglycerol caprylate) were measured after injection into mid-jejunum of fasted male SPRD rats (meant SEM, n=6).

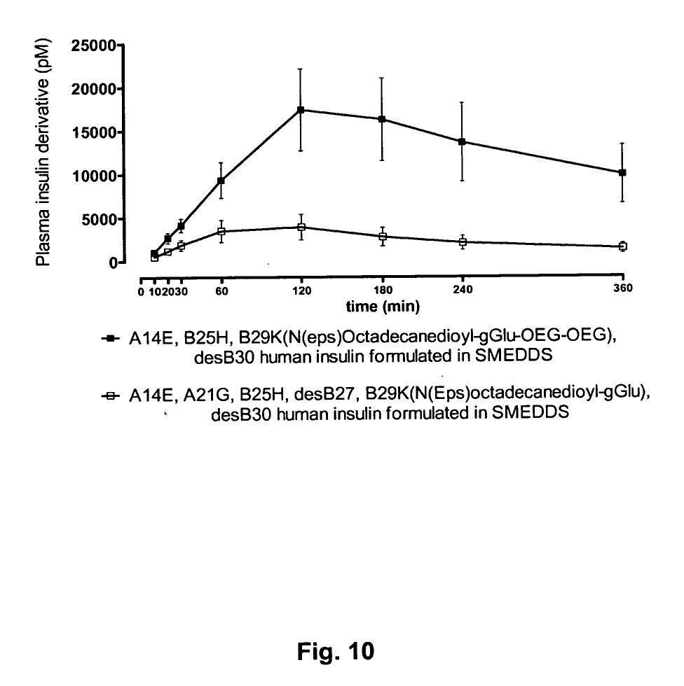

The results are shown in FIG. 10.

### Example 14

### Insulin Derivative Formulated in Different SMEDDS

Pharmakokinetic profiles were made of the insulin derivative B29K(N(eps)OctadecanedioylgGlu-OEG-OEG) A14E B25H desB30 human insulin (60 nmol/kg) formulated in different SMEDDS compositions after injection into mid-jejunum of fasted male SPRD rats (mean±SEM, n=6).

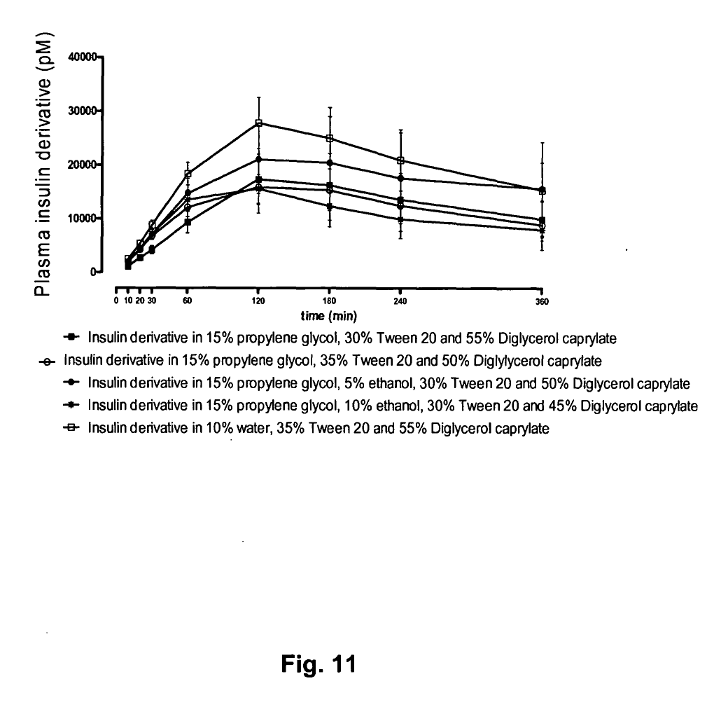

Results are illustrated in FIG. 11.

### Example 15

### Insulin Derivative Formulated in SMEDDS with Different Amounts of Water

Pharmakokinetic profiles were made of the insulin derivative B29K(N(eps)OctadecanedioylgGlu-OEG-OEG) A14E B25H desB30 human insulin (3.25 mg insulin per gram SMEDDS) formulated in a water free SMEDDS compositions and in a SMEDDS composition comprising 5% water, after injection of 0.1 ml into mid-jejunum of fasted male SPRD rats (mean±SEM, n=6).

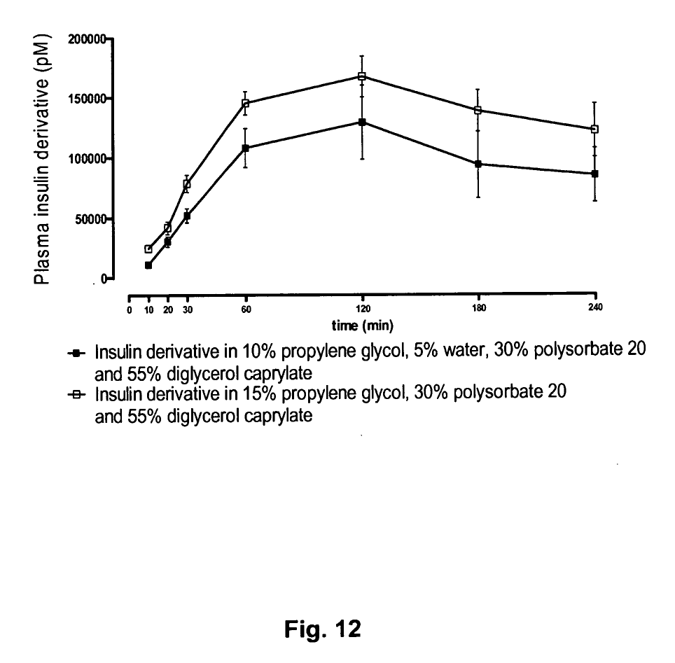

Results are illustrated in FIG. 12.

### Example 16

### Insulin Derivative Formulated in SMEDDS

Pharmakokinetic profiles were made of the insulin derivative B29K(N(eps)OctadecanedioylgGlu-OEG-OEG) A14E B25H desB30 human insulin (30 nmol/kg) formulated in SMEDDS compositions comprising propylene glycol, Tween 20 and diglycerol caprylate, after injection into mid-jejunum of fasted male SPRD rats (mean±SEM, n=6).

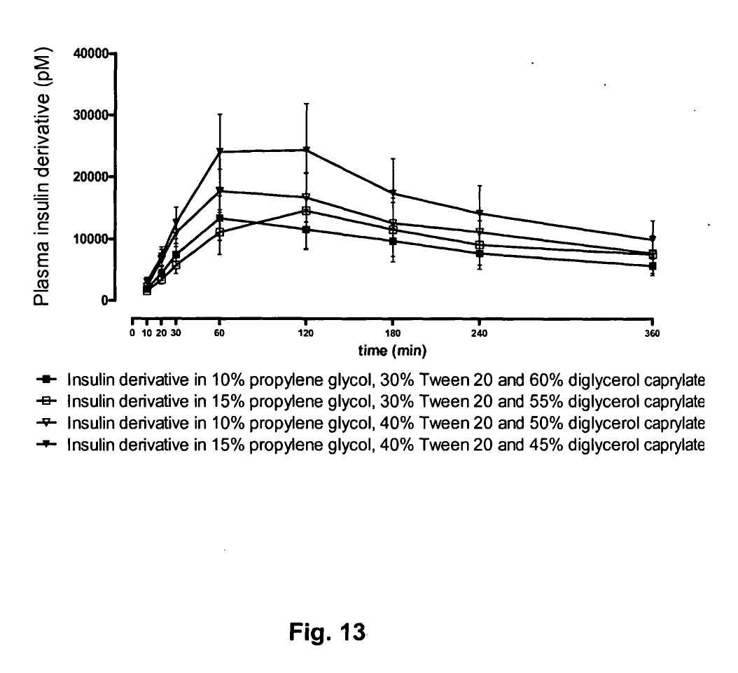

Results are illustrated in FIG. 13.

### Example 17

### Insulin Derivatives Formulated in SMEDDS Compositions

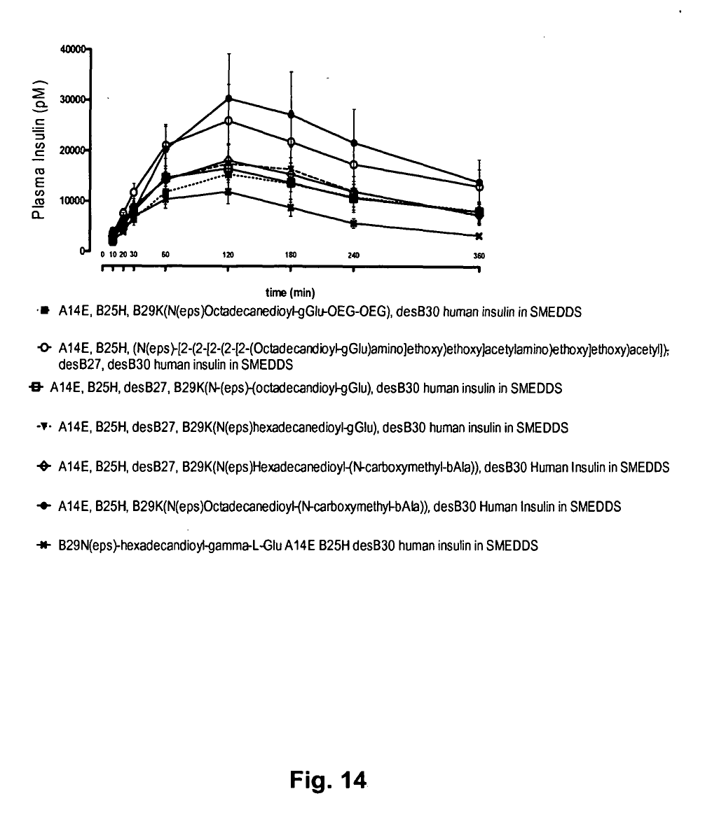

Different insulin derivatives (a, b, c, d, e, f and g, 30 nmol/kg) were each formulated in a SMEDDS composition comprising 15% propylene glycol, 30% Tween 20 and 55% diglycerol caprylate. Pharmakokinetic profiles were made after injection into mid-jejunum of fasted male SPRD rats (mean±SEM, n=6). The results are illustrated in FIG. 14.

Preparation of composition: The pH of an aqueous solution comprising the insulin derivative was adjusted to pH 7 to 8, and the solution was freeze dried. The freeze dried insulin was dissolved in propylene glycol, then diglycerol caprylate was added under agitation and in a final step Tween 20 was added under agitation at room temperature (RT). The final formulations resulted in clear homogenous SMEDDS compositions.

Insulin derivatives tested:
a) A14E, B25H, B29K(N(eps)Octadecanedioyl-gGlu-OEG-OEG), desB30 human insulin formulated in SMEDDS
b) A14E, B25H, (N(eps)-[2-(2-[2-(2-[2-(OctadecandioylgGlu)amino]ethoxy)ethoxy]acetylamino)ethoxy]ethoxy)acetyl]), desB27, desB30 human insulin in SMEDDS
c) A14E, B25H, desB27, B29K(N-(eps)-(octadecandioyl-gGlu), desB30 human insulin in SMEDDS
d) A14E, B25H, desB27, B29K(N(eps)hexadecanedioyl-gGlu), desB30 human insulin in SMEDDS
e) A14E, B25H, desB27, B29K(N(eps)Hexadecanedioyl-(N-carboxymethyl-bAla)), desB30 Human Insulin in SMEDDS
f) A14E, B25H, B29K(N(eps)Octadecanedioyl-(N-carboxymethyl-bAla)), desB30 Human Insulin in SMEDDS
g) B29N(eps)-hexadecandioyl-gamma-L-Glu A14E B25H desB30 human insulin in SMEDDS

### Pharmacological Methods

### Method of Injection Intraintestinally (Jejunum) Rat for PK Studies

Anaesthetized rats were dosed intraintestinally (into jejunum) with the insulin (derivative) peptide. Plasma concentrations of the employed compounds as well as changes in blood glucose were measured at specified intervals for 4 hours post-dosing. Pharmacokinetic parameters were subsequently calculated using WinNonLin.

Male Sprague-Dawley rats (Taconic), weighing 250-300 g, fasted for ˜18 h were anesthetized.

The anesthetized rat was placed on a homeothermic blanket stabilized at 37° C. A 20 cm polyethylene catheter mounted a 1-ml syringe was filled with insulin formulation or vehicle. A 4-5 cm midline incision was made in the abdominal wall. The catheter was gently inserted into mid-jejunum ˜50 cm from the caecum by penetration of the intestinal wall. If intestinal content was present, the application site was moved ±10 cm. The catheter tip was placed approx. 2 cm inside the lumen of the intestinal segment and fixed without the use of ligatures. The intestines were carefully replaced in the abdominal cavity and the abdominal wall and skin were closed with autoclips in each layer. At time 0, the rats were dosed via the catheter, 0.4 ml/kg of test compound or vehicle.

Blood samples for the determination of whole blood glucose concentrations were collected in heparinised 10 μl capillary tubes by puncture of the capillary vessels in the tail tip. Blood glucose concentrations were measured after dilution in 500 μl analysis buffer by the glucose oxidase method using a Biosen autoanalyzer (EKF Diagnostic Gmbh, Germany). Mean blood glucose concentration courses (mean±SEM) were made for each compound. Samples were collected for determination of the plasma insulin peptide concentration. 100 μl blood samples were drawn into chilled tubes containing EDTA. The samples were kept on ice until centrifuged (7000 rpm, 4° C., 5 min), plasma was pipetted into Micronic tubes and then frozen at 20° C. until assay. Plasma concentrations of the insulin analogs were measured using a LOCI assay.

Blood samples were drawn at t=−10 (for blood glucose only), at t=−1 (just before dosing) and at specified intervals for 4 hours post-dosing.

Plasma concentration-time profiles were analysed by a non-compartmental pharmacokinetics analysis using WinNonlin Professional (Pharsight Inc., Mountain View, Calif., USA).

Calculations were performed using individual concentration-time values from each animal.

1. A liquid pharmaceutical composition comprising at least one insulin peptide, at least one semi-polar protic organic solvent and at least two non-ionic surfactants with HLB above 10, wherein the composition does not contain oil or any other lipid component or surfactant with an HLB below 7.

2. A pharmaceutical composition according to claim 1, which comprises less than 10% w/w water.

3. A pharmaceutical composition according to claim 1, which is non-aqueous.

4. A pharmaceutical composition according to claim 1 wherein the composition forms a micro- or nanoemulsion after dilution in an aqueous medium.

5. A pharmaceutical composition according to claim 1, comprising two or three non-ionic surfactants with HLB above 10, wherein the remaining ingredients are other excipients than surfactants.

6. A pharmaceutical composition according to claim 1, wherein the semi-polar protic organic solvent is a protic solvent with a dielectricity constant in the range of 20-50.

7. A pharmaceutical composition according to claim 1, wherein the semi-polar protic organic solvent is glycerol or propylene glycol.

8. A pharmaceutical composition according to claim 1, which is in the form of a solution.

9. A pharmaceutical composition according to claim 1, wherein one or more of said non-ionic surfactants comprise a medium chain fatty acid group such as C8 fatty acids (caprylates), C10 fatty acids (caprates) or C12 fatty acids (laurates).

10. A pharmaceutical composition according to claim 1, wherein one or more of said non-ionic surfactants are selected from the group consisting of Labrasol (also named Caprylocaproyl Macrogolglycerides), Tween 20 (also named Polysorbate 20 or Polyethylene glycol sorbitan monolaurate), Tween 80 (also named polysorbate 80), Diglycerol monocaprylate, Polyglycerol caprylate and Cremophor RH 40.

11. A pharmaceutical composition according to claim 1, wherein the semi-polar protic organic solvent is present in the amount from about 1% to about 15%.

12. A pharmaceutical composition according to claim 1, wherein the insulin peptide is an insulin analogue which has an acyl moiety attached to the insulin analogue,
wherein the acyl moiety has the general formula I:
Acy-AA1n-AA2m-AA3p- (I),
wherein
n is 0 or an integer in the range from 1 to 3;
m is 0 or an integer in the range from 1 to 10;
p is 0 or an integer in the range from 1 to 10;
Acy is a fatty acid or a fatty diacid comprising from about 8 to about 24 carbon atoms;
AA1 is a neutral linear or cyclic amino acid residue;
AA2 is an acidic amino acid residue;
AA3 is a neutral, alkyleneglycol-containing amino acid residue
and wherein the order by which AA1, AA2 and AA3 appears in the formula can be interchanged independently.

13. A method of producing a pharmaceutical composition according to claim 1, wherein the method comprises the steps:
a) The insulin is dehydrated at a target pH which is at least one pH unit from the pI of the polypeptide in aqueous solution,
b) the dehydrated insulin is dissolved in the semi polar protic solvent,
c) at least two non ionic surfactants with an HLB above 10 are added together or stepwise under agitation,
d) encapsulation of the liquid formulation into soft capsules or filling into hard capsules,
e) optional enteric coating of the softcapsules or hardcapsules.

14. A pharmaceutical composition according to claim 1 for use as a medicament.

15. A method for treatment of hyperglycemia comprising oral administration of an effective amount of a pharmaceutical composition as defined in claim 1.
